SEQUENCE LISTING (55 sequences)

SEQ ID NO: 1 - protein, 17 aa, Artificial Sequence
  Synthetic peptide.
LEPGDTIIFEANGNLIA

SEQ ID NO: 2 - protein, 17 aa, Artificial sequence
  Synthetic peptide.
IIFEANGNLIAPWYAFA

SEQ ID NO: 3 - protein, 17 aa, Artificial sequence
  Synthetic peptide.
GNLIAPWYAFALSRGFG

SEQ ID NO: 4 - protein, 17 aa, Artificial sequence
  Synthetic peptide.
SSFERFEIFPKESSWPN

SEQ ID NO: 5 - protein, 17 aa, Artificial sequence
  Synthetic peptide.
LEPGDTIIFEANGNLIA

SEQ ID NO: 6 - protein, 17 aa, Artificial sequence
  Synthetic peptide.
SSFERFEIFPKESSWPN

SEQ ID NO: 7 - protein, 17 aa, Artificial sequence
  Synthetic peptide.
LEPGDTIIFEANGNLIA

SEQ ID NO: 8 - protein, 17 aa, Artificial sequence
  Syntehtic peptide.
LEPGDTIIFEANGNLIA

SEQ ID NO: 9 - protein, 17 aa, Artificial sequence
  Syntehtic peptide.
EKEVLVLWGVHHPPNIG

SEQ ID NO: 10 - protein, 17 aa, Artificial sequence
  Synthetic peptide.
VSVVSSHYSRRFTPEIA

SEQ ID NO: 11 - protein, 17 aa, Artificial sequence
  Syntehtic peptide.
EKEVLVLWGVHHPPNIG

SEQ ID NO: 12 - protein, 17 aa, Artificial sequence
  Synthetic peptide.
VSVVSSHYSRRFTPEIA

SEQ ID NO: 13 - protein, 17 aa, Artificial sequence
  Synthetic peptide.
SSFERFEIFPKESSWPN

SEQ ID NO: 14 - protein, 17 aa, Artificial sequence
  Synthetic peptide.
SRGFGSGIITSNAPMDE

SEQ ID NO: 15 - protein, 17 aa, Artificial sequence
  Synthetic peptide.
EQLSSVSSFERFEIFPK

SEQ ID NO: 16 - protein, 17 aa, Artificial sequence
  Synthetic peptide.
SRGFGSGIITSNAPMDE

SEQ ID NO: 17 - protein, 15 aa, Artificial sequence
  Synthetic peptide.
ECEKHSQPWQVLVAS

SEQ ID NO: 18 - protein, 15 aa, Artificial sequence
  Synthetic peptide.
DMSLLKNRFLRPGDD

SEQ ID NO: 19 - protein, 15 aa, Artificial sequence
  Synthetic peptide.
PEEFLTPKKLQCVDL

SEQ ID NO: 20 - protein, 15 aa, Artificial sequence
  Synthetic peptide.
HPEDTGQVFQVSHSF

SEQ ID NO: 21 - protein, 20 aa, Artificial sequence
  Synthetic peptide.
LQCVDLHVISNDVCAQVHPQ

SEQ ID NO: 22 - protein, 20 aa, Artificial sequence
  Synthetic peptide.
GVLQGITSWGSEPCALPERP

SEQ ID NO: 23 - protein, 15 aa, Artificial sequence
  Synthetic peptide.
QWVLTAAHCIRNKSV

SEQ ID NO: 24 - protein, 15 aa, Artificial sequence
  Synthetic peptide.
HSLFHPEDTGQVFQV

SEQ ID NO: 25 - protein, 15 aa, Artificial sequence
  Synthetic peptide.
PALGTTCYASGWGSI

SEQ ID NO: 26 - protein, 15 aa, Artificial sequence
  Synthetic peptide.
VISNDVCAQVHPQKV

SEQ ID NO: 27 - protein, 15 aa, Artificial sequence
  Synthetic peptide.
ITSWGSEPCALPERP

SEQ ID NO: 28 - protein, 15 aa, Artificial sequence
  Synthetic peptide.
QWVLTAAHCIRNKSV

SEQ ID NO: 29 - protein, 15 aa, Artificial sequence
  Synthetic peptide.
HSLFHPEDTGQVFQV

SEQ ID NO: 30 - protein, 15 aa, Artificial sequence
  Synthetic peptide.
PALGTTCYASGWGSI

SEQ ID NO: 31 - protein, 15 aa, Artificial sequence
  Synthetic peptide.
VISNDVCAQVHPQKV

SEQ ID NO: 32 - protein, 15 aa, Artificial sequence
  Synthetic peptide.
ITSWGSEPCALPERP

SEQ ID NO: 33 - protein, 15 aa, Artificial sequence
  Synthetic peptide.
CGGVLVHPQWVLTAA

SEQ ID NO: 34 - protein, 15 aa, Artificial sequence
  Synthetic peptide.
TAAHCIRNKSVILLG

SEQ ID NO: 35 - protein, 15 aa, Artificial sequence
  Synthetic peptide.
CIRNKSVILLGRHSL

SEQ ID NO: 36 - protein, 15 aa, Artificial sequence
  Synthetic peptide.
PALGTTCYASGWGSI

SEQ ID NO: 37 - protein, 15 aa, Artificial sequence
  Synthetic peptide.
LTPKKLQCVDLHVIS

SEQ ID NO: 38 - protein, 15 aa, Artificial sequence
  Synthetic peptide.
CGGVLVHPQWVLTAA

SEQ ID NO: 39 - protein, 15 aa, Artificial sequence
  Synthetic peptide.
TAAHCIRNKSVILLG

SEQ ID NO: 40 - protein, 15 aa, Artificial sequence
  Synthetic peptide.
CIRNKSVILLGRHSL

SEQ ID NO: 41 - protein, 15 aa, Artificial sequence
  Synthetic peptide.
PALGTTCYASGWGSI

SEQ ID NO: 42 - protein, 15 aa, Artificial sequence
  Synthetic peptide.
LTPKKLQCVDLHVIS

SEQ ID NO: 43 - protein, 15 aa, Artificial sequence
  Synthetic peptide.
CGGVLVHPQWVLTAA

SEQ ID NO: 44 - protein, 15 aa, Artificial sequence
  Synthetic peptide.
TAAHCIRNKSVILLG

SEQ ID NO: 45 - protein, 15 aa, Artificial sequence
  Synthetic peptide.
CIRNKSVILLGRHSL

SEQ ID NO: 46 - protein, 15 aa, Artificial sequence
  Synthetic peptide.
PALGTTCYASGWGSI

SEQ ID NO: 47 - protein, 15 aa, Artificial sequence
  Synthetic peptide.
LTPKKLQCVDLHVIS

SEQ ID NO: 48 - protein, 15 aa, Artificial sequence
  Synthetic peptide.
ILLGRMSLFMPEDTG

SEQ ID NO: 49 - protein, 15 aa, Artificial sequence
  Synthetic peptide.
QVFQVSHSFPHPLYD

SEQ ID NO: 50 - protein, 15 aa, Artificial sequence
  Synthetic peptide.
NDLMLLRLSEPAELT

SEQ ID NO: 51 - protein, 15 aa, Artificial sequence
  Synthetic peptide.
PALGTTCVASGMGSI

SEQ ID NO: 52 - protein, 15 aa, Artificial sequence
  Synthetic peptide.
GSIEPEEFLTPDQMK

SEQ ID NO: 53 - protein, 13 aa, Artificial sequence
  Synthetic peptide.
KKLQCVQLHVISM

SEQ ID NO: 54 - protein, 15 aa, Artificial sequence
  Synthetic peptide.
GPLVCNGVLQGITSM

SEQ ID NO: 55 - protein, 15 aa, Artificial sequence
  Synthetic peptide.
GVLQGITSMGSEPCA

(19) United States
(12) Patent Application Publication
(10) Pub. No.: US 2012/0121592 A1
(43) Pub. Date: May 17, 2012

(54) Targeting Antigens to Human Dendritic Cells Via DC-Asialoglycoprotein Receptor to Produce IL-10 Regulatory T-Cells

(75) Inventors: SangKon Oh, Baltimore, MD (US); Dapeng Li, Dallas, TX (US); Jacques F. Banchereau, Montclair, NJ (US); Sandra Zurawski, Midlothian, TX (US); Gerard Zurawski, Midlothian, TX (US)

(73) Assignee: BAYLOR RESEARCH INSTITUTE, Dallas, TX (US)

(21) Appl. No.: 13/269,951

(22) Filed: Oct. 10, 2011

Related U.S. Application Data
(60) Provisional application No. 61/392,910, filed on Oct. 13, 2010.

(51) Int. Cl. A61K 39/395; A61P 31/00; A61P 35/00; C12N 5/0783; A61P 37/04

(52) U.S. Cl. 424/134.1; 435/375

(57) ABSTRACT

Compositions and methods for targeting protein antigens to human DCs via DC-asialoglycoprotein receptor (DC-ASGPR) are disclosed herein. The DC-ASGPR carries an intracellular tyrosine-based and dileucine motif, resulting in the generation of such IL-10 Tregs both in vitro and in vivo. The methods of the present invention can be used for designing vaccines against autoimmune diseases where autoantigens are defined, such as type 1 diabetes and multiple sclerosis.

### CROSS-REFERENCE TO RELATED APPLICATIONS

This application is a non-provisional application of U.S. provisional patent application No. 61/392,910 filed on Oct. 13, 2010 and entitled “Targeting Antigens to Human Dendritic Cells via DC-Asialoglycoprotein Receptor to produce IL-10 Regulatory T Cells” the entire contents of which is incorporated herein by reference.

### STATEMENT OF FEDERALLY FUNDED RESEARCH

This invention was made with U.S. Government support awarded by the National institutes of Health (NIH)/National Institute of Allergy and Infectious Diseases (NIAID) under Contract No. U19 AI057234. The government has certain rights in this invention.

### TECHNICAL FIELD OF THE INVENTION

The present invention relates in general to immune regulation, and more particularly, to targeting protein antigens to human dendritic cells (DCs) via DC-Asialoglycoprotein receptor (DC-ASGPR) to generate IL-10 producing Regulatory T-cells.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection with the regulation of immune responses to self and foreign antigens, and limiting immune pathology associated with both infections and autoimmunity.

U.S. Patent Application Publication No. 2008/0206262 (Banchereau et al. 2008) includes compositions and methods for making and using anti DC-ASGPR antibodies that can, e.g., activate DCs and other cells. The method of the invention comprises the step of isolating and purifying a DC-ASGPR-specific antibody or fragment thereof to which an antigen is attached that forms an antibody-antigen complex, wherein the antigen is processed and presented by a dendritic cell that has been contacted with the antibody-antigen complex. The DC-ASGPR-specific antibody or fragment thereof is bound to one half of a Coherin/Dockerin pair and an antigen is bound to the complementary half of the Coherin/Dockerin pair to form a complex.

U.S. Patent Application Publication No. 2008/0241170 (Zurawski and Banchereau, 2008) includes compositions and methods for increasing the effectiveness of antigen presentation using a DCIR-specific antibody or fragment thereof to which an antigen is attached that forms an antibody-antigen complex, wherein the antigen is processed and presented by a dendritic cell that has been contacted with the antibody-antigen complex.

### SUMMARY OF THE INVENTION

The present invention describes compositions and methods for targeting protein antigens to human DCs via DC-asialoglycoprotein receptor (DC-ASGPR), which carries an intracellular tyrosine-based and dileucine motif, resulting in the generation of such IL-10 Tregs both in vitro and in vivo.

In one embodiment the present invention includes compositions and methods for generating one or more of antigen-specific regulatory (Tregs) comprising: isolating one or more human dendritic cells (DCs) from a subject; loading one or more antigens into the one or more DCs with an anti-DC-asialoglycoprotein receptor (DC-ASGPR) specific antibody or binding fragment thereof conjugated or fused to the one or more antigens to form antigen-loaded. DCs; and contacting the antigen-loaded DCs with one or more naïve T-cells, wherein the antigen-loaded DCs stimulate the proliferation of antigen-specific Tregs. In one aspect, the one or more antigens comprise peptides or proteins. In another aspect, the peptide is a foreign or a self-antigen. In another aspect, the peptide triggers an allergic, or asthmatic response. In another aspect, the one or more antigens comprise a bacterial, a viral, a fungal, a protozoan or a cancer protein. In another aspect, the antigens comprise HA-1, PSA, or combinations and modifications thereof. In yet another aspect, the antigen-specific Tregs are IL-10 secreting Tregs. In another aspect, the dendritic cells are used for a prophylaxis, a treatment, amelioration of symptoms of one or more self-antigen mediated autoimmune diseases, multiple sclerosis, influenza, or cancer. In another aspect, the autoimmune diseases are selected from the group consisting of allergies; asthma; Coeliac disease; diabetes mellitus type 1 (IDDM); systemic lupus erythematosus (SLE); Sjögren's syndrome; Churg-Strauss Syndrome; Hashimoto's thyroiditis; Graves' disease; idiopathic thrombocytopenic purpura; graft rejection; multiple sclerosis; psoriasis; and rheumatoid arthritis (RA). In another aspect, the antibody is made by a hybridoma cell contained in ATCC Deposit No. PTA-10248.

Another embodiment of the present invention includes a vaccine composition against one or more autoantigen mediated autoimmune diseases comprising an anti-DC-asialoglycoprotein receptor (ASGPR) specific antibody or binding fragment thereof conjugated to, or fused to, one or more autoantigens and one or more optional pharmaceutically acceptable adjuvants, wherein the vaccine composition generates, enhances the production or both of one or more autoantigen specific, IL-10 secreting regulatory T-cells (Tregs). In one aspect, the autoimmune diseases are selected from the group consisting of allergies; asthma; Coeliac disease; diabetes mellitus type 1 (IDDM); systemic lupus erythematosus (SUE); Sjögren's syndrome; Churg-Strauss Syndrome; Hashimoto's thyroiditis; Graves' disease; idiopathic thrombocytopenic purpura; graft rejection; multiple sclerosis; psoriasis; and rheumatoid arthritis (RA). In another aspect, the Tregs are autoantigen-specific IL-10 Tregs. In another aspect, the vaccine is administered orally, parenterally, or intra-nasally. In another aspect, the one or more antigens comprise peptides; proteins; lipid; carbohydrate; nucleic acid; and combinations thereof. In another aspect, the composition binds to and activates dendritic cells that activate the IL-10 secreting Tregs. In another aspect, the antibody is made by a hybridoma cell contained in ATCC Deposit No. PTA-10248.

Yet another embodiment of the present invention includes a method for treating, for prophylaxis, or for amelioration of symptoms of a cancer in a subject comprising the steps of: identifying the subject in need for the treatment, prophylaxis or the amelioration of symptoms against prostate cancer; administering a therapeutically effective amount of a pharmaceutical composition or a vaccine in an amount sufficient to treat, for the prophylaxis, or amelioration of the symptoms, wherein the composition comprises: a recombinant fusion protein of an anti-DC-asialoglycoprotein receptor (ASGPR) specific antibody or binding fragment thereof conjugated or fused to one or more cancer specific antigens and one or more optional pharmaceutically acceptable excipients or adjuvants. In one aspect, the one or more antigens comprise peptides or proteins. In another aspect, the peptide is a foreign or a self-antigen. In one aspect, the cancer specific antigens are peptides selected from tumor associated antigens are selected from CEA; prostate; prostate specific antigen (PSA); HER-2/neu; BAGE; GAGE; MAGE 1-4; 6 and 12; MIX (Mucin) (e.g.; MUC-1; MUC-2; etc.); GM2 and GD2 gangliosides; ras; myc; tyrosinase; MART (melanoma antigen); MARCO-MART; cyclin B1; cyclin D; Pmel 17 (gp100); GnT-V intron V sequence (N-acetylglucoaminyltransferase V intron V sequence); Prostate Ca psm; prostate serum antigen (PSA); PRAME (melanoma antigen); β-catenin; MUM-1-B (melanoma ubiquitous mutated gene product); GAGE (melanoma antigen) 1; BAGE (melanoma antigen) 2-10; c-ERB2 (Her2/neu); EBNA (Epstein-Barr Virus nuclear antigen) 1-6; gp75; human papilloma virus (HPV) E6 and E7; p53; lung resistance protein (LRP); Bcl-2; and Ki-67, in another aspect, the antibody is made by a hybridoma cell contained in ATCC Deposit No. PTA-10248.

Yet another embodiment includes a method for treating, for prophylaxis or for amelioration of symptoms of a pathogen in a subject comprising the steps of identifying the subject in need for the treatment, the prophylaxis, or the amelioration of symptoms against the pathogen; administering a therapeutically effective amount of a pharmaceutical composition or a vaccine in an amount sufficient to treat, for the prophylaxis or amelioration of the symptoms, wherein the composition comprises: a recombinant fusion protein of an anti-DC-asialoglycoprotein receptor (ASGPR) specific antibody or binding fragment thereof conjugated or fused to one or more pathogenic antigens and one or more optional pharmaceutically acceptable excipients or adjuvants. In one aspect, the vaccine is administered orally, parenterally, or intra-nasally. In another aspect, the vaccine generates, enhances a level, or both of one or more of pathogen-specific regulatory T-cells (Tregs). In one aspect, the antibody is made by a hybridoma cell contained in ATCC Deposit No. PTA-10248.

Yet another embodiment of the present invention includes a method for treating, for prophylaxis, or amelioration of symptoms of autoantigen mediated autoimmune diseases in a subject comprising the steps of: identifying the subject in need of the treatment, the prophylaxis, or the amelioration of the symptoms of the autoimmune disease; and administering a therapeutically effective amount of vaccine comprising a recombinant fusion protein of an anti-DC-asialoglycoprotein receptor (ASGPR) specific antibody or binding fragment thereof conjugated or fused to one or more autoantigens and one or more optional pharmaceutically acceptable adjuvants, wherein the vaccine composition generates, enhances the production, or both of one or more autoantigen specific regulatory T-cells (Tregs). In one aspect, the autoimmune diseases are selected from the group consisting of allergies; asthma; Coeliac disease; diabetes mellitus type 1 (IDDM); systemic lupus erythematosus (SLE); Sjögren's syndrome; Churg-Strauss Syndrome; Hashimoto's thyroiditis; Graves' disease; idiopathic thrombocytopenic purpura; graft rejection; multiple sclerosis; psoriasis; and rheumatoid arthritis (RA). In another aspect; the autoimmune disease is diabetes mellitus type 1 (IDDM). In another aspect, the Tregs are autoantigen-specific Tregs that secrete IL-10. In another aspect, the vaccine is administered orally, parenterally, or intra-nasally. In another aspect, the one or more antigens comprise peptides; proteins; lipid; carbohydrate; nucleic acid; and combinations thereof. In another aspect, the antibody is made by a hybridoma cell contained in ATCC Deposit No. PTA-10248.

Yet another embodiment of the present invention includes a pharmaceutical composition for generating self-antigen specific regulatory (Tregs) comprising: a recombinant fusion protein of an anti-DC-asialoglycoprotein receptor (ASGPR) specific antibody or binding fragment thereof conjugated or fused to one or more self-antigens; and one or more optional pharmaceutically acceptable excipients or adjuvants. In one aspect, the composition is used for a prophylaxis; a treatment; amelioration of symptoms of one or more autoantigen mediated autoimmune diseases; multiple sclerosis; influenza; or cancer. In another aspect, the self-antigen is selected from antigens that cause allergies; asthma; Coeliac disease; diabetes mellitus type 1 (IDDM); systemic lupus erythematosus (SLE); Sjögren's syndrome; Churg-Strauss Syndrome; Hashimoto's thyroiditis; Graves' disease; idiopathic thrombocytopenic purpura; graft rejection; multiple sclerosis; psoriasis; and rheumatoid arthritis (RA). In another aspect, the antibody is made by a hybridoma cell contained in ATCC Deposit No. PTA-10248. Yet another embodiment is a composition comprising antigen-specific Tregs made by the methods of the present invention.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B: In vivo and in vitro DCs express LOX-1 and DC-ASGPR: FIG. 1A expression of LOX-1 (upper panels) and DC-ASGPR (lower panels) on the surface of blood myeloid DCs (mDCs) (Lin−HLA−DR+CD11c+CD123−), plasmacytoid DCs (pDCs) (Lin−HLA−DR+CD11c−CD123+), CD14+ monocytes and CD3+ T-cells, FIG. 1B surface expression of LOX-1 (upper panels) and DC-ASGPR (lower panels) on monocyte-derived IFNDCs and IL-4DCs;

FIGS. 2A-2D: Immunifluorescent staining of healthy human skin sections: FIG. 2A frozen sections (5 mm) were stained with a, DAPI, anti-HLA-DR, anti-CD1c and anti-LOX-1 mAbs labeled with fluorescents, FIG. 2B DAPI, anti-HLA-DR, anti-CD1c and anti-DC-ASGPR mAbs labeled with fluorescents, FIG. 2C DAPI and control mAbs, FIG. 2D Summary of data generated with skins from two healthy donors. Each dot represents data from one section;

FIGS. 3A-3E: Recombinant fusion proteins of anti-DC surface receptor mAbs and antigens: FIG. 3A reducing SDS-PAGE analysis of purified recombinant fusion proteins (Lane 1: Molecular weight markers, Lane 2: anti-LOX-1, Lane 3: Anti-DC-ASGPR, Lane 4: Control IgG4-HA1, Lane 5: anti-LOX-1-HA1, Lane 6: anti-DC-ASGPR-HA1, Lane 7: Control IgG4-PSA, Lane 8: anti-LOX-1-PSA, and Lane 9: anti-DC-ASGPR-PSA, FIGS. 3B and 3C binding of HA1 and PSA fusion proteins to IFNDCs, FIGS. 3D and 3E CD4+ T-cell proliferation induced by IFNDCs loaded with 1 mg/ml recombinant fusion proteins, 1 mg/ml mAbs, or none. Data are representative of five-independent experiments using cells from different healthy donors;

FIG. 4: Influenza viral HA1 delivered to DCs via LOX-1 or DC-ASGPR results in HA1-specific IFNg (Interferon gamma)-producing CD4+ T-cell responses, 5×10³ IFNDCs were loaded with 1 μg/ml anti-LOX-1-HA1 or anti-DC-ASGPR-HA1 fusion proteins, and then incubated overnight. Purified autologous CD4+ T-cells (2×10⁵) labeled with CFSE were co-cultured for seven days. CD4+ T-cells were restimulated with peptide pools (0.1 μM of each peptide, 11-12 peptides per clusters, 17-mers overlapping by 11 amino acids) in the presence of BFA. Cells were then stained for CD4 and intracellular IFNg. Two separate studies show similar results;

FIGS. 5A-5G: Antigen targeting to DCs via DC-ASGPR generate Th1-originated antigen-specific IL-10-producing CD4+ T-cells: FIG. 5A frequency of HA1-derived peptide-specific IFNγ-expressing CD4⁺ T-cells elicited by IFNDCs loaded with 1 μg/ml recombinant fusion proteins, FIG. 5B frequency of HA1-derived peptide-specific IL-10-expressing CD4⁺ T-cells. Peptide HA1₂₈₀₋₂₉₆ was tested as a negative control in FIGS. 5A and 5B. Four-independent studies showed similar results in FIGS. 5A and 5B, FIG. 5C cytokine levels in the culture supernatants. Each line represents the data from a single run, FIG. 5D CD4⁺ T-cells expanded by DCs loaded anti-LOX-1-HA1 or anti-DC-ASGPR-HA1 were stimulated with PMA (50 ng/ml)/inonomycin (1 μg/ml) for 4 h, and then stained for intracellular IFNγ and IL-10, FIG. 5E Frequency of PSA-derived IL-10-producing CD4⁺ T-cells elicited by IFNDCs loaded with 1 μg/ml recombinant fusion protein, FIG. 5F cytokine levels in culture supernatants. Error bars represent mean±SD of triplicate assay. Three-independent studies were performed with PSA-derived 59 peptides. Only peptides that resulted in positive responses in all three experiments are presented. Control peptides for each donor are indicated, FIG. 5G CD4⁺ T-cells expanded by DCs loaded anti-LOX-1-HA1 or anti-DC-ASGPR-HA1 were stimulated with PMA (50 ng/ml)/inonomycin (1 μg/ml) for 4 h, and then stained for intracellular IFNγ and IL-10. More than four independent studies showed similar results in FIGS. 5D and 5G;

FIG. 6: HA1 delivered to DCs via DC-ASGPR results in enhanced HA1-specific IL-10-producing CD4+ T-cell responses: 5×10³ DCs were loaded with 1 mg/ml anti-LOX-1-HA1 or anti-DC-ASGPR-HA1, and incubated overnight. Autologous CD4+ T-cells (2×10⁵) were co-cultured for seven days. On day 9, CD4+ T-cells were restimulated with 1 mM peptides loaded IFNDCs for 48 h. IL-10 levels in the culture supernatants were measured by Luminex. Studies using the same peptide clusters were performed three times in triplicate assays. Values are the average of 12 data points. Gray color indicates the average ±SD acquired with no peptide;

FIG. 7: Expression levels of Foxp3, PD-1 and CTLA-4 on HA1₂₅₀₋₂₆₆-specific IL-10-producing CD4+ T-cells;

FIG. 8: PSA delivered to DCs via DC-ASGPR results in enhanced PSA-specific IL-10-producing CD4+ T-cell responses: 5×10³ DCs were loaded with 1 mg/ml anti-LOX-1-PSA or anti-DC-ASGPR-PSA, and incubated overnight. Autologous CD4+ T-cells (2×10⁵) were co-cultured for seven days. On day 9, CD4+ T-cells were restimulated with 1 mM peptides loaded IFNDCs for 48 h. IFNg and IL-10 levels in the culture supernatants were measured by Luminex, Error bars represent mean±SD of triplicate assay. Three-independent studies were performed with PSA-derived 59 peptides. Only peptides that resulted in positive responses in all three experiments are presented. Control peptides for each donor are indicated;

FIG. 9: Expression levels of Foxp3, PD-1 and CTLA-4 on PSA30-44-specific (B) IL-10 producing CD4+ T-cells. Three independent studies resulted in similar data;

FIGS. 10A-10C: Specialized function of DC-ASGPR for generating antigen-specific IL-10-producing CD4+ T-cell: 5×10³ IFNDCs were loaded with 1 mg/ml recombinant fusion proteins of PSA or pooled peptide (10 mM). After overnight incubation, CFSE-labeled autologous CD4+ T-cells (2×10⁵) were co-cultured for seven days. FIG. 10A CD4+ T-cells were restimulated with 1 mM peptides PSA₁₀₂₋₁₁₆ or control peptides (PSA₈₂₋₉₆) loaded IFNDCs. After 48 h, IL-10 and IFNg in the culture supernatants were measured by Luminex. Data from five independent studies are summarized, FIG. 10B CD4+ T-cells were restimulated with 1 mM peptides PSA₁₀₂₋₁₁₆ or control peptides (PSA₈₂₋₉₆) in the presence of BFA for staining intracellular IL-10, FIG. 10C summary of three independent studies of FIG. 10B. P values were calculated with student t-test;

FIGS. 11A-11D: IFNDCs are more potent than other APCs for generating antigen-specific CD4+ T-cells that secrete IFNg and IL-10. 5×10³ APCs were loaded with 1 mg/ml recombinant fusion proteins of HA1. After overnight incubation, CFSE-labeled autologous CD4+ T-cells (2×10⁵) were co-cultured for seven days. On day 9, CD4+ T-cells were restimulated with 1 mM HA1₂₅₀₋₂₆₆ or control peptides HA1₂₈₀₋₂₉₆ loaded IFNDCs in the presence of brefeldin A. Cells were stained for intracellular IL-10 and IFNg. Data from four independent studies are summarized. P values were calculated with student t-test;

FIGS. 12A and 12B: Peptide-loaded IFNDCs are more efficient for inducing CD4+ T-cells to express intracellular IFNg and IL-10 than other APCs loaded with the same peptides. 5×10³ IFNDCs were loaded with 1 mg/ml anti-LOX-1-HA1 (FIG. 12A) or anti-DC-ASGPR-HA1 (FIG. 12B). After overnight incubation, CFSE-labeled autologous CD4+ T-cells (2×10⁵) were co-cultured for seven days. On day 10, CD4+ T-cells were restimulated with APCs loaded with 5 mM. HA1₂₅₀₋₂₆₆ or control peptides HA1₂₈₀₋₂₉₆ in the presence of brefeldin A. Cells were then stained for intracellular IL-10 and IFNg. Data from four (FIG. 12A) and three (FIG. 12B) independent studies are summarized. P values were calculated with student t-test;

FIGS. 13A-13D: CD4+ T-cells generated with anti-DC-ASGPR-PSA and anti-DC-ASGPR-HA1 suppress both allogeneic and HA1-specific IFNg-producing CD4+ T-cell responses: FIG. 13A different numbers of FACS-sorted CFSElow effector cells generated with anti-DC-ASGPR-PSA (left panel) or anti-LOX-1-PSA (right panel) were co-cultured with anti-DC-ASGPR-PSA-loaded IFNDCs and newly purified allogeneic CD4⁺⁰ T-cells. Proliferation of allogeneic CD4⁺ T-cells was assessed by measuring ³[H]-thymidine uptake, FIG. 13B FACS-sorted CFSE^{low} effector cells generated with anti-DC-ASGPR-PSA were co-cultured anti-DC-ASGPR-loaded IFNDCs and newly purified allogeneic CD4⁺ T-cells in the presence of anti-IL-10 and anti-IL-10R antibodies. Effector: responding cell ratio was 1:2. Proliferation of allogeneic CD4⁺ T-cells was assessed by measuring ³[H]-thymidine uptake. Three independent studies with quadruplicate assays showed similar results in FIGS. 13A and 13B. Representative data from one run presented, FIG. 13C FACS-sorted CFSE^{low} effector cells generated with anti-DC-ASGPR-HA1 were co-cultured with IFNDCs loaded with HA1₂₅₀₋₂₆₆ in the upper wells. Newly purified and CFSE-labeled CD4⁺ T-cells and IFNDCs loaded with anti-LOX-1-HA1 were co-cultured in the lower wells of trans-well plates. On day 6, proliferation of CD4⁺ T-cells in the lower wells were assessed by measuring CFSE-dilution. Anti-IL-10/IL-10R or control IgG were added into lower wells, FIG. 13D on day 10, the frequency of IFNγ-expressing CD4⁺ T-cells were measured. Data from five independent studies are summarized in the right panel;

FIGS. 14A-14G: Antigen targeting to DCs via DC-ASGPR induces IL-10 in DCs, and this IL-10 contributes to the generation of antigen-specific IL-10-producing CD4+ T-cells: FIG. 14A after 15 min of loading IFNDCs with 1 mg/ml recombinant proteins (anti-DC-ASGPR-PSA and anti-LOX-1-PSA) or 10 mg/ml Zymosan, cell lysate were analyzed for ERK, phospho-ERK, p38, and phospho-p38 by western blots, FIG. 14B phospho-flow cytometric analysis of ERR/p38 phosphorylation, FIG. 14C IL-10 levels in the culture supernatants of IFNDCs (1×10⁵) loaded with 1 mg/ml anti-LOX-1-PSA or anti-DC-ASGPR-PSA. Cells were incubated for 24 hours. Four independent studies showed similar results, FIG. 14D IL-10 blocking: purified CD4+ T-cells were co-cultured with IFNDCs loaded with anti-DC-ASGPR-PSA in the presence of control IgG or anti-IL-10/IL-10R antibodies. On day 9, cells were restimulated with indicated peptides (1 mM) and stained for intracellular IL-10, FIG. 14E summary of the data from four independent studies of FIG. 14D, FIG. 14F exogenous IL-10: CD4+ T-cells were co-cultured with IFNDCs loaded with anti-LOX-1-PSA in the presence or absence of 100 ng/ml IL-10. On day 9, cells were stained for intracellular IL-10, FIG. 14G summary of the data from five independent studies for FIG. 14F. Each dot represents the data from one separate study. P values were acquired by student t-test;

FIGS. 15A-15C: Blocking TGFb1 results in decreased levels of IL-10-producing antigen-specific CD4+ T-cell responses, but addition of exogenous TGFb1 did not enhance the responses. 5×10³ IFNDCs were loaded with 1 mg/ml anti-DC-ASGPR-PSA. After overnight incubation, CFSE-labeled autologous CD4+ T-cells (2×10⁵) were co-cultured for seven days in the presence of 20 mg/ml anti-TGFb1 and anti-TGFb Receptor. On day 9, CD4+ T-cells were restimulated with IFNDCs loaded with 1 mM peptides in the presence of brefeldin A. FIG. 15A cells were then stained for intracellular IL-10, FIG. 15B data from three independent studies are summarized, FIG. 15C IFNDCs loaded with anti-LOX-1-PSA and CFSE-labeled CD4+ T-cells were co-cultured in the presence of 100 ng/ml TGFb1. Cells were stained for intracellular IL-10;

FIGS. 16A and 16B: DC-ASGPR signals could prevail over LOX-1 signals: FIG. 16A 5×10³ IFNDCs loaded with 1 mg/ml of fusion proteins and purified CD4+ T-cells (2×10⁵) were co-cultured for 10 days. CD4+ T-cells were restimulated with 1 mM HA1₂₅₀₋₂₆₆ or control peptides HA1₂₈₀₋₂₉₆. IFNg and IL-10 levels were measured by the Luminex, FIG. 16B 5×10³ IFNDCs loaded with 1 mg/ml of PSA fusion proteins and purified CD4+ T-cells (2×10⁵) were co-cultured for 10 days. CD4+ T-cells were restimulated with 1 mM. PSA30-44 or control peptides. Each dot represent the data from an independent study. P values were calculated with student t-test; and

FIGS. 17A-17C: Data on non-human primates immunized with antigens (HA1 and PSA) carried by anti-DC-ASGPR mAb elicit antigen-specific IL-10-producing cellular responses: FIG. 17A expression levels of LOX-1 and DC-ASGPR in CD11c+, CD14+, and CD3+ cells in PBMCs of cynomolgus macaques. FIGS. 17B and 17C Twelve animals primed and boosted i.d. with live influenza viruses (H1N1, A/PR8/38) were divided into two groups (6 animals/group). One group of animals was immunized i.d. with 250 mg anti-LOX-1-HA1 (right arm) and 250 mg anti-LOX-1-PSA (left arm). The other group was immunized i.d. with 250 mg anti-DC-ASGPR-HA1 (right arm) and 250 mg anti-DC-ASGPR-PSA (left arm). After the second boosting with the recombinant fusion proteins, PBMCs (2×10⁵ cells/200 ml of medium/well in 96-well plates) were restimulated with 25 mM peptide pools of HA1 (FIG. 17B) or PSA (FIG. 17C), respectively. IFNg and IL-10 in the culture supernatants were measured by ELISA. Peptide pools of HIVgag were used as controls. Values presented in FIGS. 17B and 17C are after subtraction of control values. Cells were also stimulated with staphylococcal enterotoxin B (10 mg/ml) (right panels in FIGS. 17B and 17C). Statistical significance was tested by ANOVA.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “antigen” or “Ag” refer to a substance capable of eliciting an immune response, e.g., a T-cell-mediated immune response by the presentation of the antigen on Major Histocompatibility Antigen (MHC) cellular proteins and causing an antigen-specific T-cells response. In the case of a regulatory T-cell (Treg) response to the antigen is a decrease or amelioration of the immune response by other effector cells, e.g., helper T-cells (Th) and/or cytotoxic T-cells (Tc). The skilled immunologist will recognize that when discussing antigens that are processed for presentation to T-cells, the term “antigen” refers to those portions of the antigen (e.g., a peptide fragment) that is a T-cell epitope presented by MHC to the T-cell receptor. When antigen is modified by self- or auto-, this refers to self or auto antigens that are commonly present in MHC molecules but that also trigger a T-cell response. When used in the context of a B cell mediated immune response in the form of an antibody that is specific for an “antigen”, the portion of the antigen that binds to the complementarity determining regions of the variable domains of the antibody (light and heavy) the bound portion may be a linear or three-dimensional epitope. In certain cases, the antigens delivered by the vaccine or fusion protein of the present invention are internalized and processed by antigen presenting cells prior to presentation, e.g., by cleavage of one or more portions of the antibody or fusion protein.

As used herein, the term “antigenic peptide” refers to that portion of a polypeptide antigen that is specifically recognized by either B-cells and/or T-cells. B-cells respond to foreign antigenic determinants via antibody production, whereas T-lymphocytes mediate cellular immunity. Thus, antigenic peptides in a T-cell response are those parts of an antigen that are recognized by antigen-specific T-cell receptors in the context of MHC.

As used herein, the term “epitope” refers to any protein determinant capable of specific binding to an immunoglobulin or of being presented by a Major Histocompatibility Complex (MHC) protein (e.g., Class I or Class II) to a T-cell receptor. Epitopic determinants are generally short peptides 5-30 amino acids long that fit within the groove of the MHC molecule that presents certain amino acid side groups toward the T-cell receptor and has certain other residues in the groove, e.g., due to specific charge characteristics of the groove, the peptide side groups and the T-cell receptor. Generally, an antibody specifically binds to an antigen when the dissociation constant is 1 mM, 100 nM or even 10 nM.

As used herein the term “Antigen Presenting Cells” (APC) are cells that are capable of activating T-cells, and include, but are not limited to, certain macrophages, B cells and dendritic cells. “Dendritic cells” (DCs) refer to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refer to any protein for which receptors are expressed on a dendritic cell. Examples include GM-CSF, IL-1, TNF, IL-4, CD40L, CTLA4, CD28, and FLT-3 ligand.

For the purpose of the present invention, the term “vaccine composition” is intended to mean a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes, or both. As used herein the term “antigen” refers to any antigen which can be used in a vaccine, whether it involves a whole microorganism or a subunit, and whatever its nature: peptide, protein, glycoprotein, polysaccharide, glycolipid, lipopeptide, etc. They may be viral antigens, bacterial antigens, or the like; the term “antigen” also comprises the polynucleotides, the sequences of which are chosen so as to encode the antigens whose expression by the individuals to which the polynucleotides are administered is desired, in the case of the immunization technique referred to as DNA immunization. They may also be a set of antigens, in particular in the case of a multivalent vaccine composition which comprises antigens capable of protecting against several diseases, and which is then generally referred to as a vaccine combination, or in the case of a composition which comprises several different antigens in order to protect against a single disease, as is the case for certain vaccines against whooping cough or the flu, for example. The term “antibodies” refers to immunoglobulins, whether natural or partially or wholly produced artificially, e.g., recombinant. An antibody may be monoclonal or polyclonal. The antibody may, in some cases, be a member of one, or a combination immunoglobulin classes, including: IgG, IgM, IgA, IgD, and IgE.

The term “antibody” includes, but is not limited to, both naturally occurring and non-naturally occurring antibodies that are isolated and/or purified. Specifically, the term “antibody” includes polyclonal and monoclonal antibodies, and binding fragments thereof that continue to bind to antigen. Furthermore, the term “antibody” includes chimeric antibodies and wholly synthetic antibodies, and fragments thereof. Polyclonal antibodies are derived from the sera of animals immunized with the antigen. Monoclonal antibodies can be prepared using hybridoma technology (Kohler et al., Nature 256:495 (1975); Hammerling et al., in Monoclonal Antibodies and T-Cell Hybridomas, Elsevier, N.Y. pp. 563-681 (1981)), relevant portions incorporated herein by reference. Antibodies also include polyclonal antibodies, affinity-purified polyclonal antibodies, monoclonal antibodies, and antigen-binding fragments, such as F(ab′)2 and Fab proteolytic fragments. Genetically engineered intact antibodies or fragments, such as chimeric antibodies. Fv fragments, single chain antibodies and the like, as well as synthetic antigen-binding peptides and polypeptides, are also included that bind to DC-ASGPR. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Moreover, human antibodies can be produced in transgenic, non-human animals that have been engineered to contain human immunoglobulin genes as disclosed in, e.g., WIPO Publication WO 98/24893, relevant portions incorporated herein by reference.

The term “humanized antibodies” refers to chimeric antibodies that comprise constant regions from human antibodies and hybrid variable regions in which most or all of the framework sequences are from a human variable region and all or most of the CDRs are from a non-human variable region. Humanized antibodies are also referred to as chimeric or veneered antibodies and are produced by recombinant techniques and readily available starting materials. Such techniques are described, for example, in UK Patent Application GB 2,188,638 A, relevant portions incorporated herein by reference.

The terms “effective amount” or “therapeutically effective amount” refers to the amount of the subject compound that will elicit the biological or medical response of a tissue, system, animal or human that is being sought by the researcher, veterinarian, medical doctor or other clinician.

As used herein, the term “treatment” or “treating” refers to administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The term “fusion protein” refers to the expression product of two or more nucleic acid molecules that are not natively expressed together as one expression product. Fusion proteins can be made at the nucleic acid coding level by placing, in-line and in the correct coding frame, the two or more sequences of the portions of the proteins or peptides. Fusion proteins are synthesized by methods known to those of skill in the art including, e.g., solid phase protein synthesis, and by molecular techniques that permit the manipulation of DNA in vitro, including polymerase chain reaction (PCR) and oligonucleotide-directed mutagenesis. A fusion protein for use with the present invention is an immunotoxin, which includes an antigen binding portion, in this case an anti-DC-ASGPR antibody of fragment thereof and a toxin.

As used herein, the term “autoimmune disorder” refers to a disease state in which a patient's immune system recognizes a self-antigen or auto antigen in the patient's organs or tissues as foreign and becomes activated. The activated immune cells that are directed against self or auto antigens can cause damage to the target organ or tissue or can damage other organs or tissues. The dysregulated immune cells secrete inflammatory cytokines that cause systemic inflammation or they can recognize self-antigens as foreign, thereby accelerating the immune response against self-antigens. Non-limiting examples of autoimmune diseases include: rheumatoid arthritis; auto-immune or auto-inflammatory diseases of the skin; allergy; sclerosis; arteriosclerosis; multiple sclerosis; asthma; psoriasis; lupus; systemic lupus erythematosis; diabetes mellitus; myasthenia gravis; chronic fatigue syndrome; fibromyalgia; Crohn's disease; Hashimoto's thyroiditis; Grave's disease; Addison's disease; Guillian Barre syndrome; and scleroderma. “Auto-immune” refers to an adaptive immune response directed at self-antigens. “Auto-immune disease” refers to a condition wherein the immune system reacts to a “self” antigen that it would normally ignore, leading to destruction of normal body tissues. Auto-immune disorders are considered to be caused, at least, in part, by a hypersensitivity reaction similar to allergies, because in both cases the immune system reacts to a substance that it normally would ignore. Auto-immune disorders include, e.g., Hashimoto's thyroiditis; pernicious anemia; Addison's disease; type 1 (insulin dependent) diabetes; rheumatoid arthritis; systemic lupus erythematosus; Sjogren's syndrome; multiple sclerosis; myasthenia gravis; Reiter's syndrome; and Grave's disease; alopecia greata; anklosing spondylitis; antiphospholipid syndrome; auto-immune hemolytic anemia; auto-immune hepatitis; auto-immune lymphoproliferative syndrome (ALPS); auto-immune thrombocytopenic purpura (ATP); Behcet's disease; bullous pemphigoid; cardiomyopathy; celiac sprue-type dermatitis; chronic fatigue syndrome immune deficiency syndrome (CFIDS); chronic inflammatory demyelinating polyneuropathy; cicatricial pemphigold; cold agglutinin disease; limited scleroderma (CREST syndrome); Crohn's disease; Dego's disease; dermatomyositis; discoid lupus; essential mixed cryoglobulinemia; fibromyalgia-fibromyositis; Guillain-Barre syndrome; idiopathic pulmonary fibrosis; idiopathic thrombocytopenia purpura (ITP); IgA nephropathy; juvenile rheumatoid arthritis; Meniere's disease; mixed connective tissue disease; pemphigus vulgaris; polyarteritis nodosa; polychondritis; polyglancular syndromes; polymyalgia rheumatica; polymyositis; primary agammaglobulinemia; primary biliary cirrhosis; psoriasis; Raynaud's phenomenon; rheumatic fever; sarcoidosis; scleroderma; stiff-man syndrome; Takayasu arteritis; temporal arthritis/giant cell arthritis; ulcerative colitis; uveitis; vasculitis; vitiligo; and Wegener's granulomatosis.

Some autoimmune disorders are also chronic inflammatory diseases, which are generally defined as a disease process with long-term (>6 months) activation of cells that lead to inflammation. Chronic inflammation may also lead to damage of patient organs or tissues. Many diseases are chronic inflammatory disorders, but are not know to have an autoimmune basis, e.g., atherosclerosis; congestive heart failure; Crohn's disease; ulcerative colitis; polyarteritis nodosa; Whipple's disease; and primary Sclerosing Cholangitis.

Other antigenic peptides for use with the present invention include cancer peptides selected from tumor-associated antigens, e.g., autologous cancer antigens obtained from a patient. Non-limiting examples of cancer antigens include antigens from leukemias and lymphomas; neurological tumors such as astrocytomas or glioblastomas; melanoma; breast cancer; lung cancer; head and neck cancer; gastrointestinal tumors; gastric cancer; colon cancer; liver cancer; pancreatic cancer; genitourinary tumors such cervix; uterus; ovarian cancer; vaginal cancer; testicular cancer; prostate cancer or penile cancer; bone tumors; vascular tumors; or cancers of the lip; nasopharynx; pharynx and oral cavity; esophagus; rectum; gall bladder; biliary tree; larynx; lung and bronchus; bladder; kidney; brain and other parts of the nervous system; thyroid; Hodgkin's disease; non-Hodgkin's lymphoma; multiple myeloma and leukemia. In a specific aspect the composition further comprises antigenic peptides selected from tumor associated antigens are selected from CEA; prostate specific antigen (PSA); HER-2/neu; BAGE; GAGE; MAGE 1-4; 6 and 12; MUC (Mucin) (e.g., MUC-1, MUC-2, etc.); GM2 and GD2 gangliosides; ras; myc; tyrosinase; MART (melanoma antigen); MARCO-MART; cyclin B1; cyclin D; Pmel 17 (gp100); GnT-V intron V sequence (N-acetylglucoaminyltransferase V intron V sequence); Prostate Ca psm; prostate serum antigen (PSA); PRAME (melanoma antigen); β-catenin; MUM-1-B (melanoma ubiquitous mutated gene product); GAGE (melanoma antigen) 1; BAGE (melanoma antigen) 2-10; c-ERB2 (Her2/neu); EBNA (Epstein-Barr Virus nuclear antigen) 1-6; gp75; human papilloma virus (HPV) E6 and E7; p53; lung resistance protein (LRP); Bcl-2; and Ki-67.

The production and use of IL-110 secreting Tregs can also be used for the treatment of conditions such as, e.g., inflammatory bowel diseases such as ileitis, ulcerative colitis and Crohn's disease; inflammatory lung disorders such as bronchitis, oxidant-induced lung injury and chronic obstructive airway disease; inflammatory disorders of the eye, e.g.; corneal dystrophy; ocular hypertension; trachoma; onchocerciasis; retinitis; uveitis; sympathetic ophthalmitis and endophthalmitis; chronic inflammatory disorders of the gum including periodontitis; chronic inflammatory disorders of the joints; e.g.; arthritis; septic arthritis and osteoarthritis; tuberculosis arthritis; leprosy arthritis; sarcoid arthritis; disorders of the skin; e.g.; sclerodermatitis; sunburn; psoriasis and eczema; encephalomyelitis and viral or autoimmune encephalitis; autoimmune diseases including immune-complex vasculitis; and disease of the heart; e.g.; ischemic heart disease; heart failure; and cardiomyopathy caused by T-cells that are recognizing one or more self-antigens or auto-antigens.

The term “adjuvant” refers to a substance that enhances, augments or potentiates the host's immune response to a vaccine antigen.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate; phosphoroselenoate; phosphorodiselenoate; phosphoroanilothioate; phosphoranilidate; phosphoramidate; and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used in this application, the term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “in vivo” refers to being inside the body. The terms “in vitro” or “ex vivo” as used in the present application are to be understood as indicating an operation carried out in a non-living system or outside a living system.

As used herein, the term “treatment” or “treating” refers to any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes compositions and methods for generating, enhancing a level or both of one or more of antigen-specific regulatory T-cells (Tregs) by targeting protein antigens to human DCs via DC-asialoglycoprotein receptor (DC-ASGPR), which carries an intracellular tyrosine-based and dileucine motif 14, resulting in the generation of IL-10 Tregs both in vitro and in vivo.

In one embodiment, the present invention includes an anti-DC-ASGPR antibody that may be, e.g., the AB4-5.49C11.7 (HS4128), which has been deposited with the American Type Culture Collection under Deposit No. PTA-10248, in compliance with the terms of the Budapest Treaty on the International Recognition of the Deposit of Micro-organisms at the American Type Culture Collection (ATCC) 10801 University Boulevard, Manassas, Va. 20110-220.

Dendritic cell (DCs) can control immune responses, in part, by expressing toll-like receptors and lectins 10-13, 15. Human DC-lectins, such as Dectin-1, lectin-like oxidized-LDL receptor (LOX-1), and DC-specific ICAM-3-grabbing nonintegrin (DC-SIGN) can deliver intracellular signals, resulting in altered T-cell responses 10-12, 15, 16. Human DC-ASGPR possesses a tyrosine-based and dileucine motif 14, but it's biological function has not been fully studied.

Antibodies and other reagents: Monoclonal antibodies (mAbs) specific for human DC lectins, including LOX-1 and DC-ASGPR, and recombinant fusion proteins of HA1 and PSA were generated in this study. mAbs were labeled with Alexa fluor 488 or 568 (Molecular Probes) were used. Antibodies used for staining cell surface (anti-CD3, anti-CD4, anti-CD11c, anti-CD14, anti-CD19, anti-CD123, anti-CTLA-4, and anti-PD-1) were purchased from Biolegend (CA). To block IL-10, anti-IL-10 (BIIR) and anti-IL-10R (R&D systems, MN) were used. Antibodies and reagents for intracellular staining were purchased from BD Pharmingen (CA) and cells were stained following the manufacturer's protocol. In general, 0.5×106-1×106 cells were incubated with 0.5-1 mg/ml antibodies for 20-30 min on ice for both cell surface and intracellular staining. Anti-TGFb1 (1D11) and anti-TGFbR were purchased from R&D. Antibodies used for in this study were purchased from BD Biosciences. Anti-CD1c and anti-HLA-DR were from Biolegend and anti-LOX-1 was purchased from Abeam (MA). Recombinant IL-10 was purchased from R&D. IFNa, IL-4 and GM-CSF were purchased from the pharmacy at Baylor University Medical Center (TX). IL-2 and CFSE were purchased from Peprotech (NJ) and Molecular Probes (CA), respectively. HA1 and PSA peptide libraries were purchased from Mimotopes (CA).

Cells: IL-4DCs (Interleukin-4 activated or matured DC) and IFNDCs (Interferon gamma activated or matured dendritic cells) were prepared by culturing monocytes from healthy donors. Monocytes were cultured in Cellgenix (UK) medium containing 100 ng/ml GM-CSF and 50 ng/ml IL-4 (IL-4DCs) for 6 days or 100 ng/ml GM-CSF and 500 units/ml IFNa (IFNDCs) for 3 days. Total CD4+ T-cells, CD14+ monocytes, and CD19+ B cells were purified by using negative selection kits (StemCell, CA). Naïve CD4+ (CD45RA+CD45RO−) (purity>99.2%) were purified by FACS Aria (BD Biosciences, CA).

CD4+ T-cell responses. 5×10³ IFNDCs loaded with 1-2 mg/ml recombinant proteins were co-cultured with 2×10⁵ purified and CFSE-labeled CD4+ T-cells. Cell proliferation was tested by measuring CFSE dilution on day 6. On day 9 or 10, CD4+ T-cells were restimulated with 1-5 mM peptide-loaded APCs for 5 h in the presence of brefeldin A (1 ml/ml). Cells were then stained for intracellular cytokine expressions. Cytokine levels in the culture supernatants were measured using the BeadLyte cytokine assay kit (Upstate, MA) as per the manufacturer's protocol. In suppression assays using allogeneic systems, CFSElow CD4+ T-cells from the primary culture were sorted and used as effector cells. Different numbers of effector cells and newly purified CD4+ T-cells (responding cells) from the same donors were co-cultured with allogenic DCs in the presence of 10 mg/ml of anti-IL-10 and anti-IL-10R antibodies or the same concentrations of control IgG. CD4+ T-cells were pulsed with 1 mCi/well 3[H]-thymidine for the final 18 h before harvesting. 3[H]-thymidine incorporation was measured by 1450 Microbeta counter (Wallac, MA). Trans-well plates (96 well-plates inserts, Nunc, PA) were used in autologous antigen-specific suppression assays. CFSElow effector cells were generated with DCs loaded with anti-DC-ASGPR-HA1 or anti-LOX-1-HA1 and anti-DC-ASGPR-PSA or anti-LOX-1-PSA. Sorted effector cells were co-cultured with 1-5 mM peptide-loaded DCs in upper wells. CFSE-labeled purified CD4+ T-cells (responders) were co-cultured with DCs loaded with anti-LOX-1-HA1 in lower wells. Blocking or control antibodies were added into lower wells. On day 6, proliferation of CD4+ T-cells in lower wells was assessed by measuring CFSE-dilution using flow cytometry. On day 9, CD4+ T-cells were restimulated with peptide-loaded DCs and stained for intracellular IFNg (IFN-gamma) expressions.

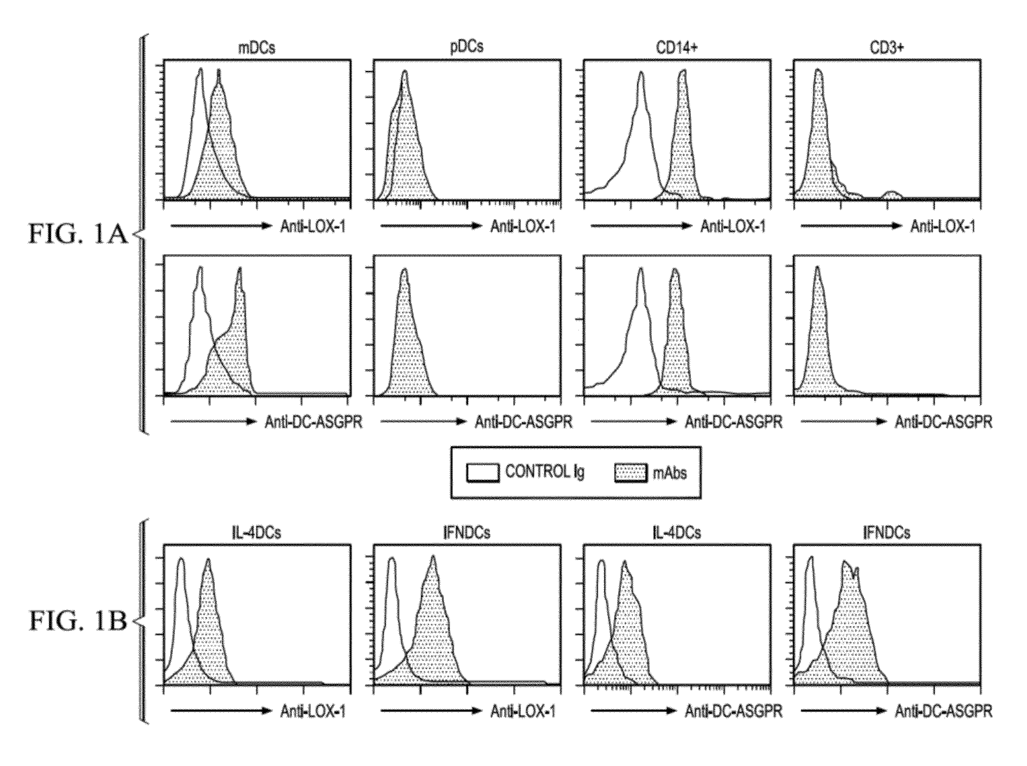

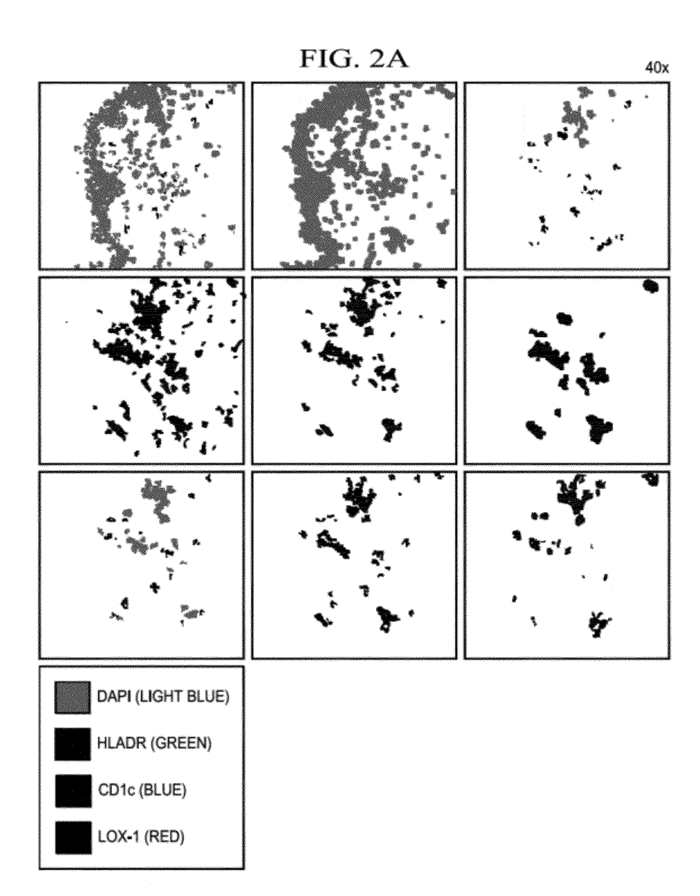

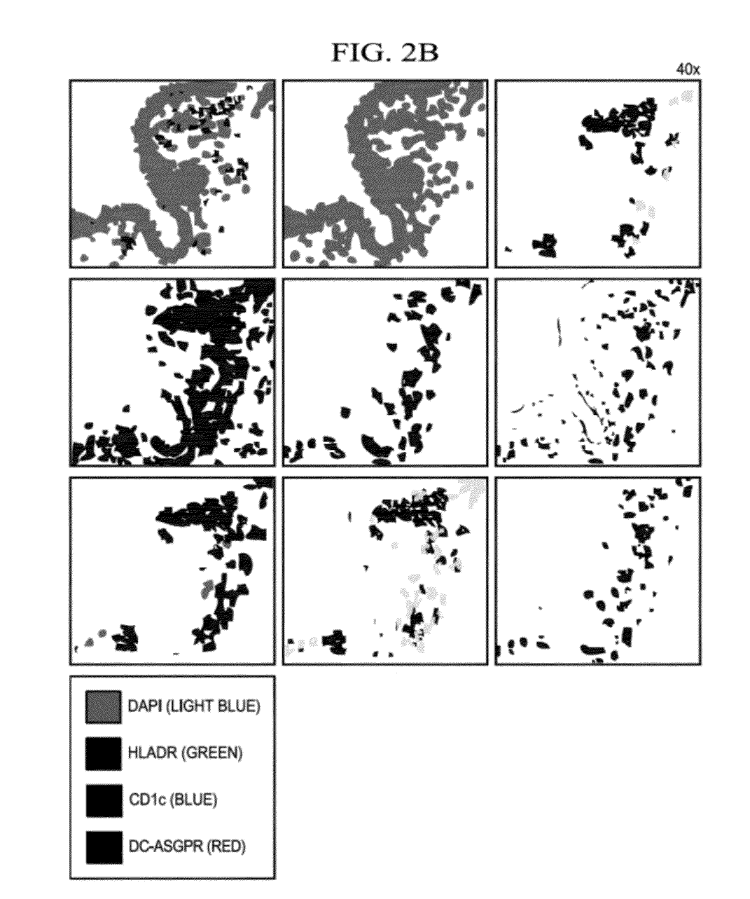

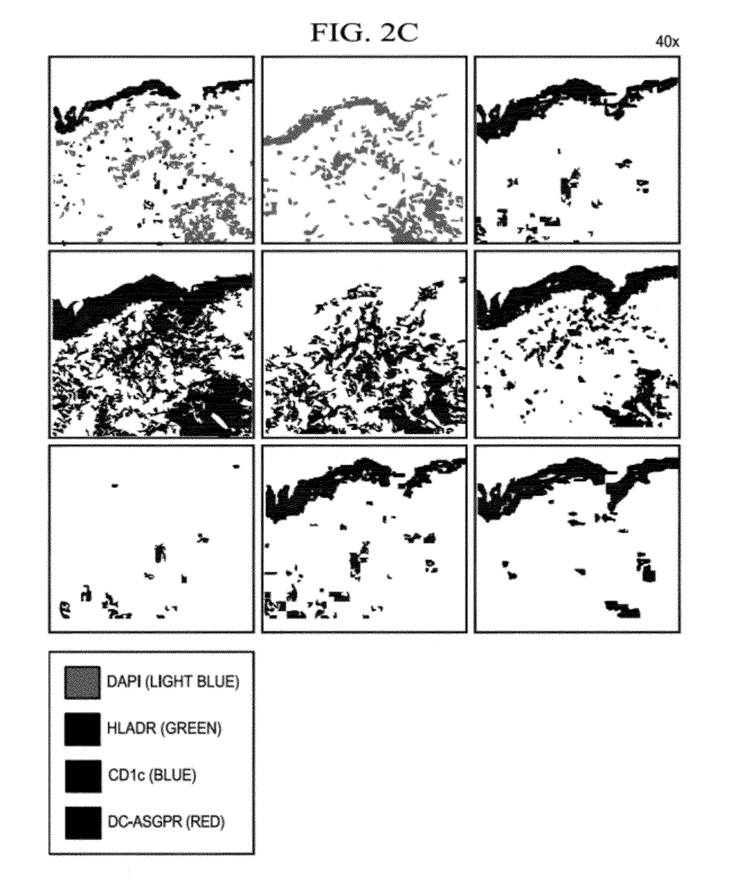

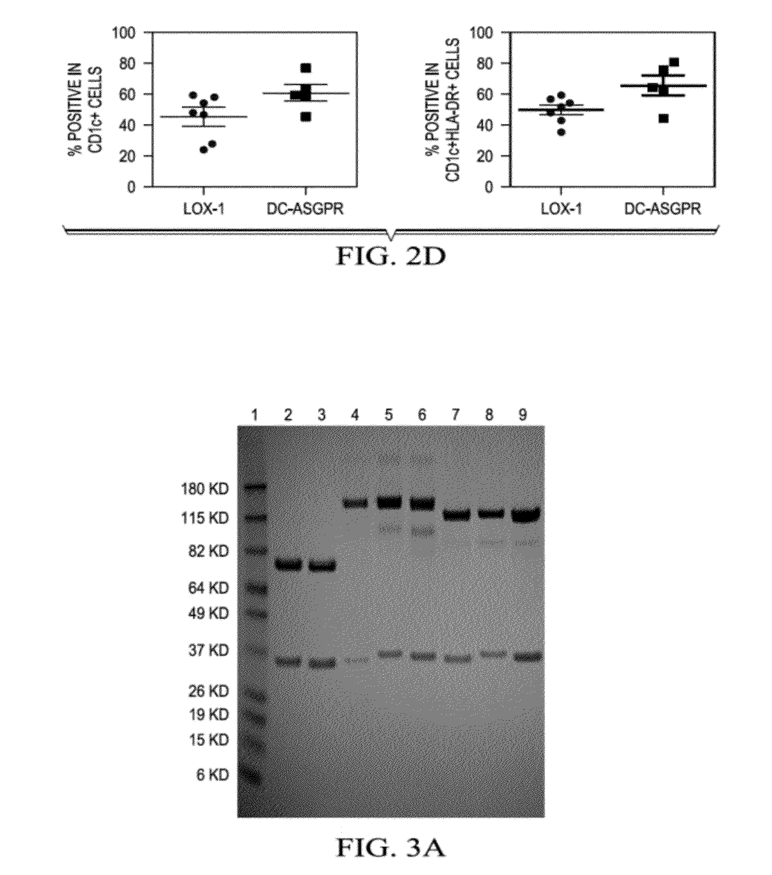

To target antigens to DCs via DC-ASGPR, monoclonal antibodies (mAbs) specific for human DC-ASGPR were generated. Anti-LOX-1 mAb was generated as a control. Both anti-LOX-1 (IgG2a: clone 15C4) and anti-DC-ASGPR (IgG2a: clone 49C11) mAbs bound to myeloid DCs (mDCs) and CD14+ monocytes, but not plasmacytoid DCs (pDCs) (FIG. 1A). In vitro cultured monocyte-derived IL-4DCs and IFNDCs also expressed both lectins (FIG. 1B). In human skin, cells expressing LOX-1 and DC-ASGPR were localized mainly in the dermis (FIGS. 2A-2D).

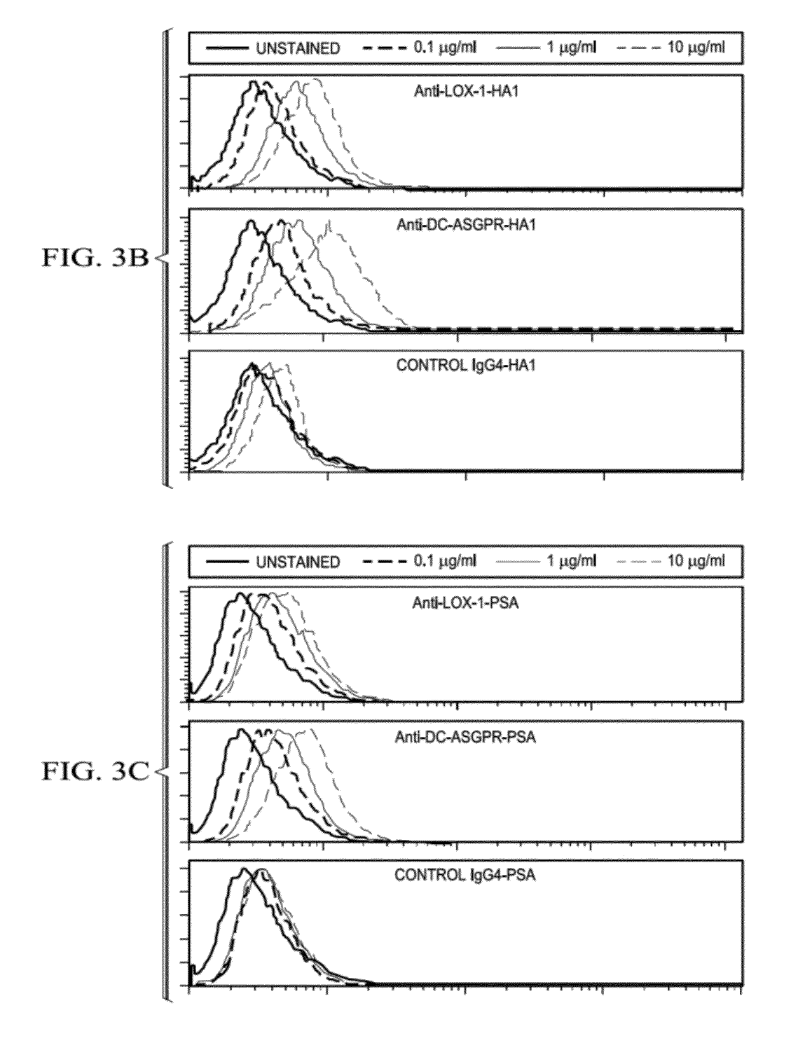

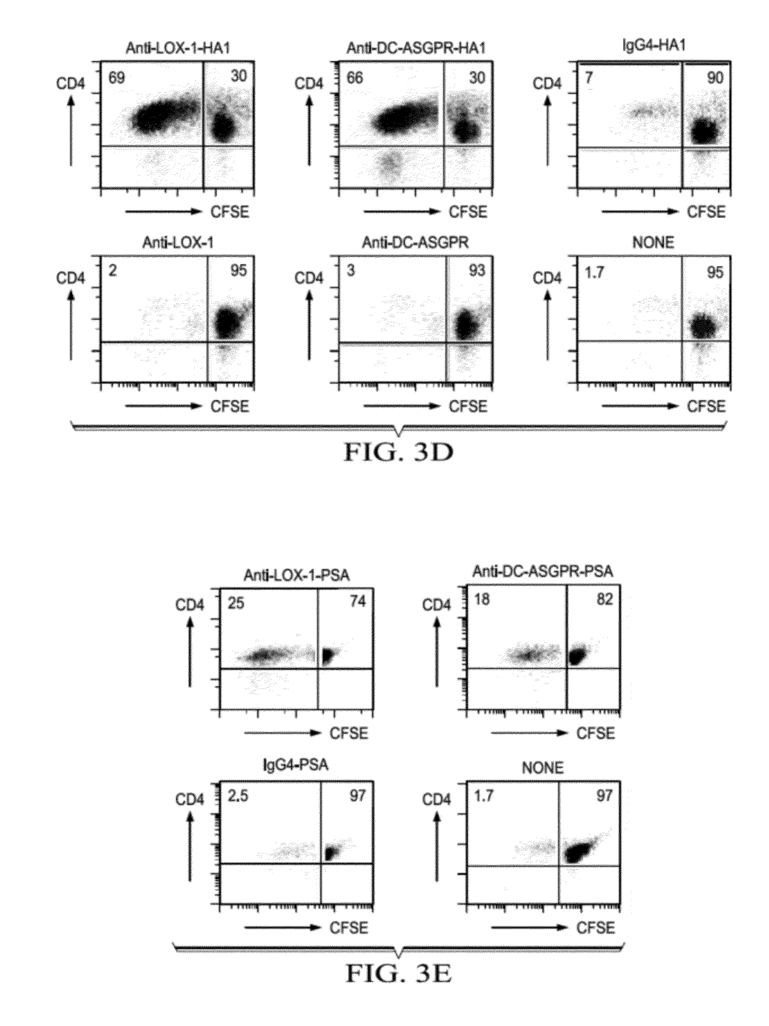

HA1 (HA1 subunit of influenza virus A/PR/8/34, H1N1) and prostate-specific antigen (PSA) were chosen as foreign and self-antigens, respectively. Recombinant mAbs fused to HA1 (anti-LOX-1-HA1, anti-DC-ASGPR-HA1, and IgG4-HA1) or PSA (anti-LOX-1-PSA, anti-DC-ASGPR-PSA, and IgG4-PSA) were generated as mouse variable region-human IgG4k chimeras with two site mutations (S228P and L235E) 17 (FIG. 3A). Both anti-LOX-1-HA1 and anti-DC-ASGPR-HA1 bound to IFNDCs more efficiently than did IgG4-HA1 (FIGS. 3B AND 3C). Anti-LOX-1-HA1 and anti-DC-ASGPR-HA1 (1 mg/ml) induced greater CD4+ T-cell proliferation (>66%) than did IgG4-HA1 (7%) or unloaded-DCs (1.7%) (FIG. 3D). Anti-LOX-1-PSA (25%) and anti-DC-ASGPR-PSA (18%) also induced greater CD4+ T-cell proliferation than did IgG4-PSA (2.5%) (FIG. 3E).

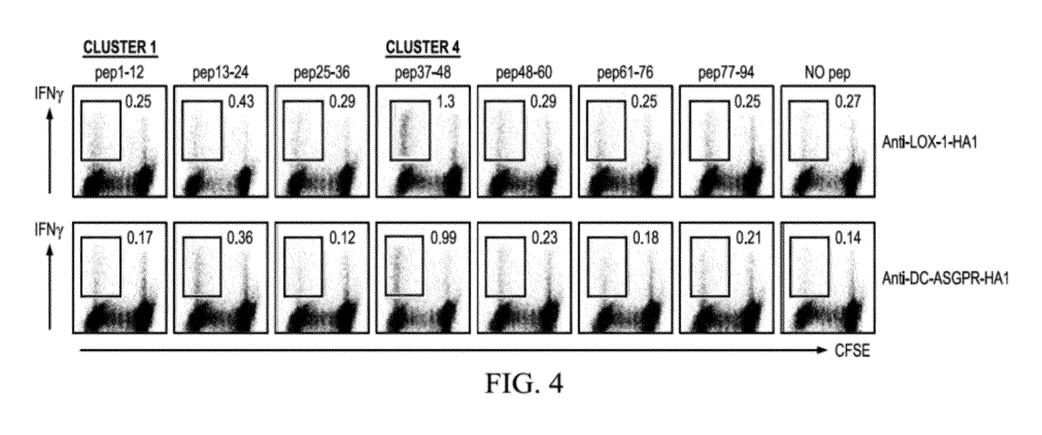

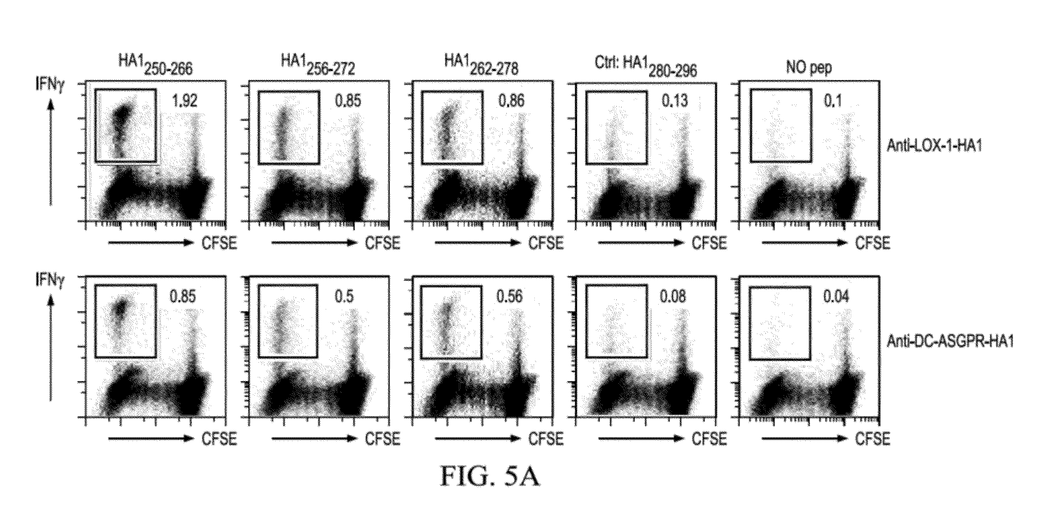

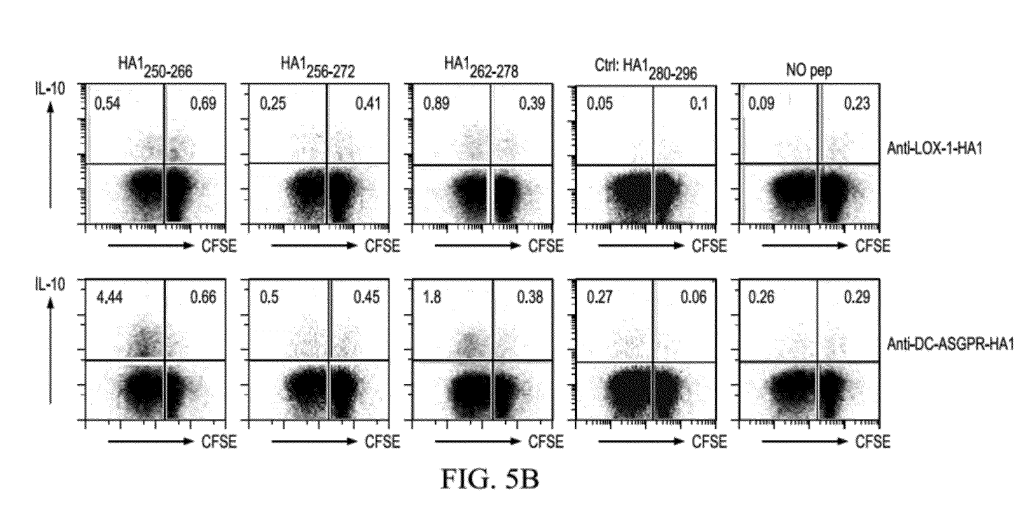

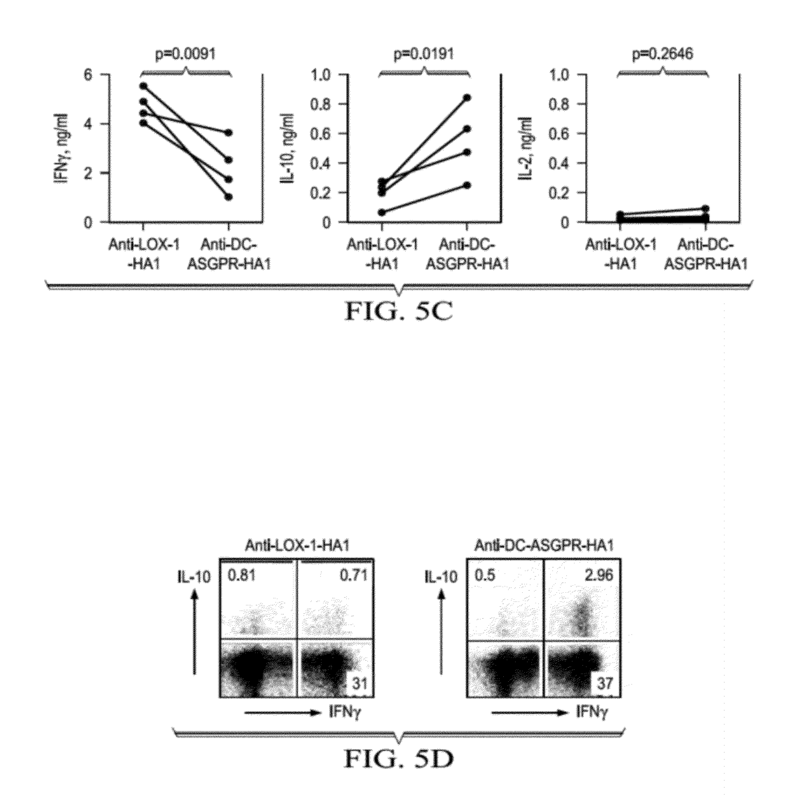

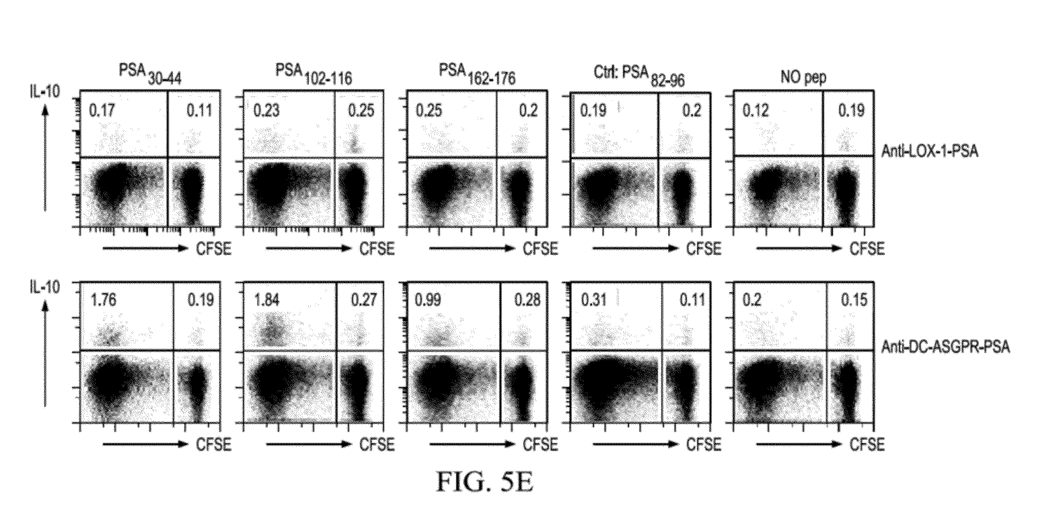

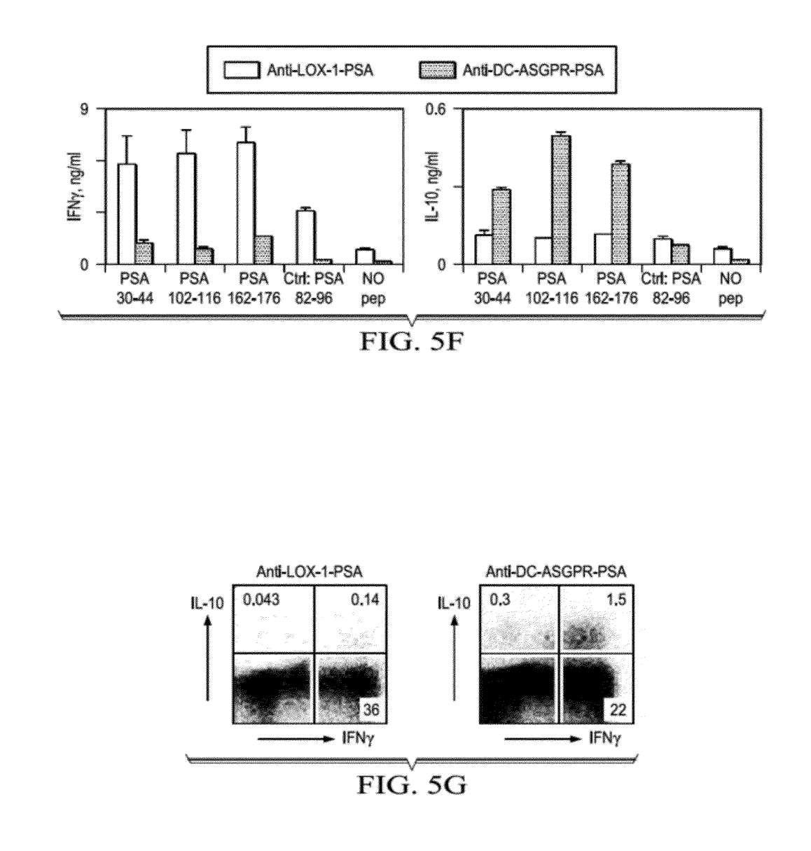

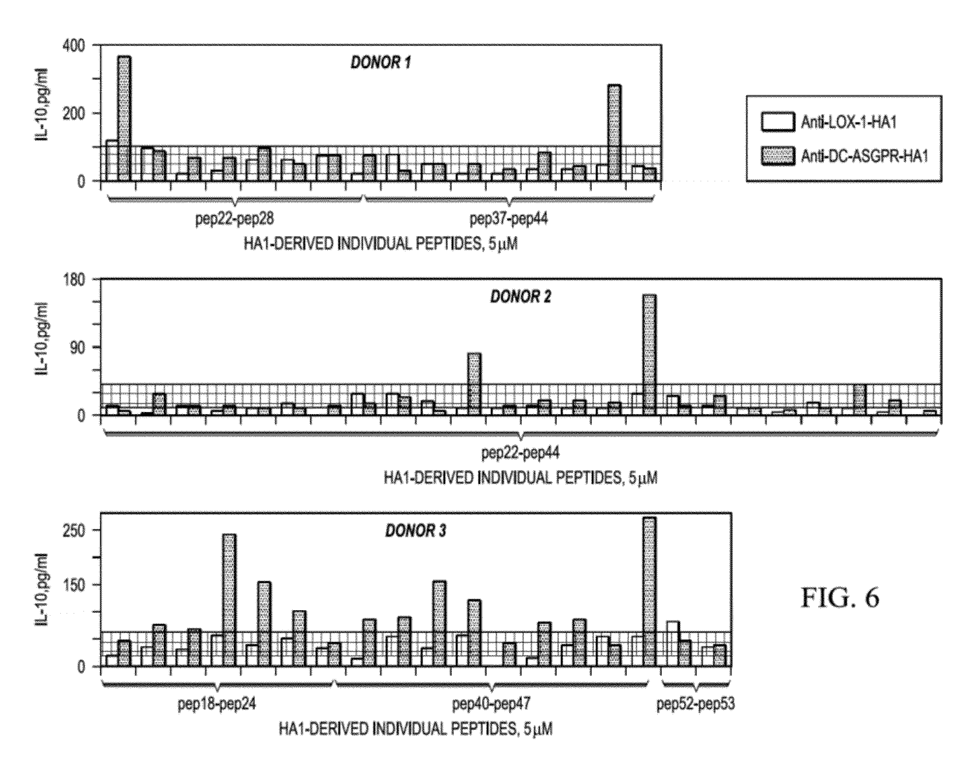

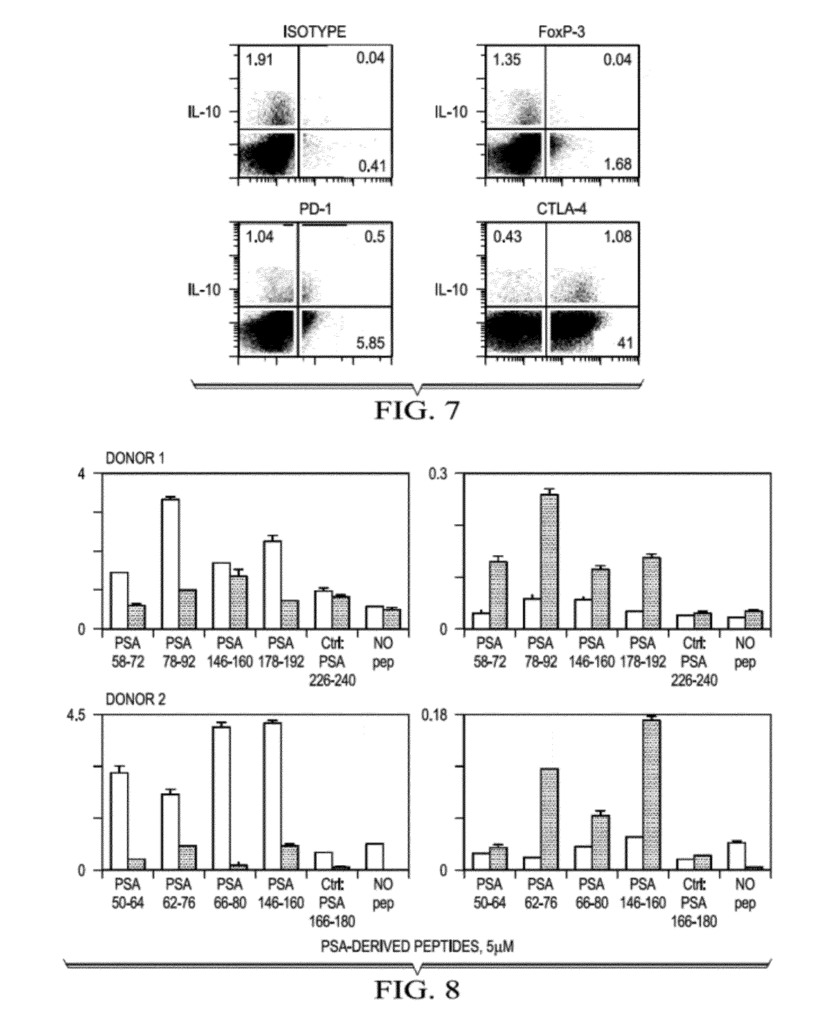

The antigen specificity of proliferating CD4+ T-cells was first tested by measuring intracellular IFNg expression during restimulation with seven HA1-derived peptide clusters (FIG. 4). Individual peptides in cluster 4 were further analyzed (FIG. 5A). Both anti-LOX-1- and anti-DC-ASGPR-HA1 resulted in HA1₂₅₀₋₂₆₆-, HA1₂₅₆₋₂₇₂-, and HA1₂₆₂₋₂₇₈-specific IFNg-producing CD4+ responses. Interestingly, anti-DC-ASGPR-HA1 resulted in greater numbers of HA1-specific IL-10-expressing CD4+ T-cells cells than did anti-LOX-1-HA1 (FIG. 5B). HA1₂₅₀₋₂₆₆-specific CD4+ T-cells expanded with anti-DC-ASGPR-HA1 secreted higher levels of IL-10 (p<0.02) and lower levels of IFNg (p<0.02) than CD4+ T-cell expanded with anti-LOX-1-HA1 (FIG. 5F). Studies using cells from three additional healthy donors also showed the enhanced IL-10-producing CD4+ T-cell responses by anti-DC-ASGPR-HA1 (FIG. 6). Only low levels of IL-2 and other cytokines (IL-4 and IL-5<50 pg/ml, not shown) were detected. PMA/ionomycin stimulates anergic Th1 cells to produce IFNg 18. FIG. 5D shows that the majority of IL-10-producing CD4+ T-cells express IFNg when stimulated with PMA/ionomycin (FIG. 5D), confirming their Th1 origin 6. They did not express Foxp3 (FIG. 7), but >60% expressed CTLA-4. A small fraction (≈30%) expressed low levels of PD-1. Taken together, HA1 delivered to DCs via DC-ASGPR results in enhanced HA1-specific IL-10-producing Foxp3-CD4+ T-cell responses of Th1 origin. HA1-derived peptides determined in this study are summarized in Table 1.

TABLE 1
Predicted binding scores of HA-1 derived peptides to corresponding HLA class II of the
healthy donors tested.

| | | | Binding scores |
|---|---|---|---|
| Donors | Class II types | Peptides tested in this study | (ABR scores) |
| Donor | HLA-DRB01-13 | HA1₅₀₋₂₆₆ LEPGDTIIFEANGNLIA (SEQ ID NO: 1) | (1000000) |
| | | HA1₅₆₋₂₇₂ IIFEANGNLIAPWYAFA (SEQ ID NO: 2) | (9879.0) |
| | | HA1₆₂₋₂₇₈ GNLIAPWYAFALSRGFG (SEQ ID NO: 3) | (83821.7) |
| | HLA-DRB3* | NA | NA |
| | HLA-DQB1*06 | NA | NA |
| Donor 1 | HLA-DRB01-07 | HA1₂₆₋₁₄₂ SSFERFEIFPKESSWPN (SEQ ID NO: 4) | (5796.6) |
| | | HA1₅₀₋₂₆₆ LEPGDTIIFEANGNLIA (SEQ ID NO: 5) | (1000000) |
| | HLA-DRB01-15 | HA1₂₆₋₁₄₂ SSFERFEIFPKESSWPN (SEQ ID NO: 6) | (461455.1) |
| | | HA1₅₀₋₂₆₆ LEPGDTIIFEANGNLIA (SEQ ID NO: 7) | (1000000) |
| | HLA-DRB5* | HA1₅₀₋₂₆₆ LEPGDTIIFEANGNLIA (SEQ ID NO: 8) | (1000000) |
| | HLA-DRB4* | NA | NA |
| | HLA-DQB1* | NA | NA |
| Donor 2 | HLA-DRB01-01 | HA1₁₈₆₋₂₀₂ EKEVLVLWGVHHPPNIG (SEQ ID NO: 9) | (261.9) |
| | | HA1₂₁₅₋₂₃₁ VSVVSSHYSRRFTPEIA (SEQ ID NO: 10) | (1109.7) |
| | HLA-DRB01-08 | HA1₁₈₆₋₂₀₂ EKEVLVLWGVHHPPNIG (SEQ ID NO: 11) | (2634.8) |
| | | HA1₂₁₅₋₂₃₁ VSVVSSHYSRRFTPEIA (SEQ ID NO: 12) | (30673.0) |
| | HLA-DQB1* | NA | NA |
| Donor 3 | HLA-DRB01-07 | HA1₁₂₆₋₁₄₂ SSFERFEIFPKESSWPN (SEQ ID NO: 13) | (5796.6) |
| | | HA1₂₇₄₋₂₉₀ SRGFGSGIITSNAPMDE (SEQ ID NO: 14) | (2723.8) |
| | HLA-DRB01-11 | HA1₁₂₀₋₁₃₆ EQLSSVSSFERFEIFPK (SEQ ID NO: 15) | (18064.8) |
| | | HA1₂₇₄₋₂₉₀ SRGFGSGIITSNAPMDE (SEQ ID NO: 16) | (85130.7) |
| | HLA-DRB3* | NA | NA |
| | HLA-DRB4* | NA | NA |
| | HLA-DQB1* | NA | NA |

1. Peptides were predicted by the algorithm in the web: (http://tools.immuneepitope.org/analyze/cgi-bin/mhc_II_binding.py). Maximum and minimal ABR score is 1000000 and 0, respectively.
2. NA: alleles are not available in the algorithm.

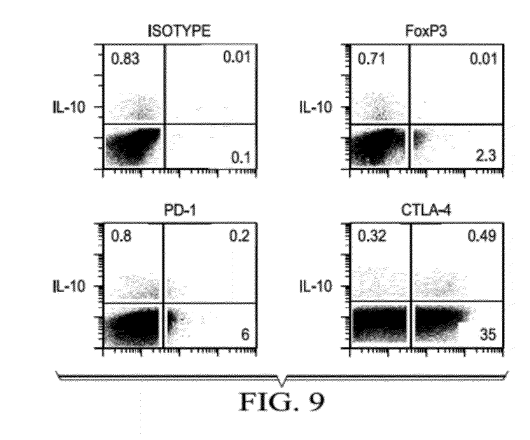

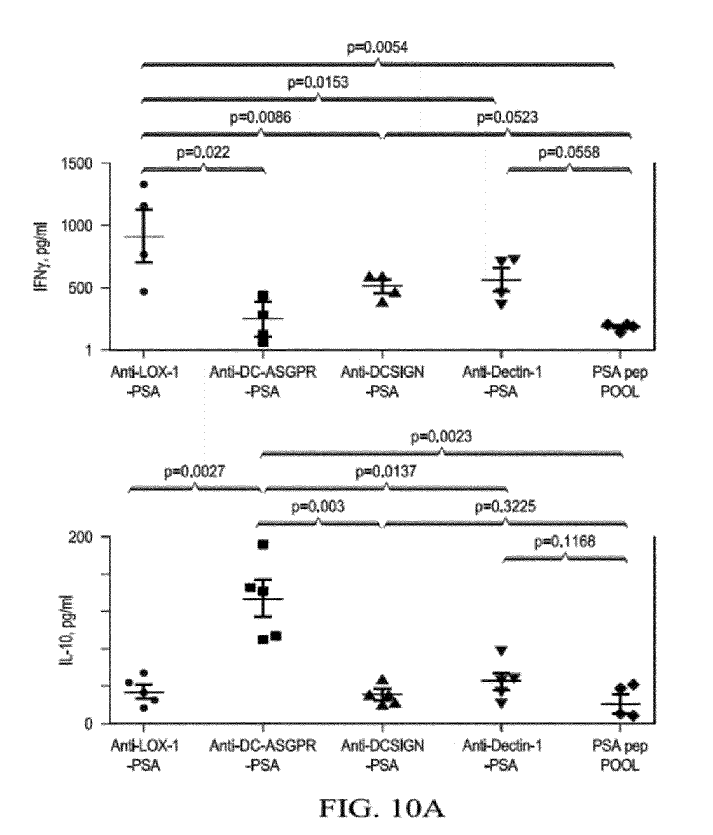

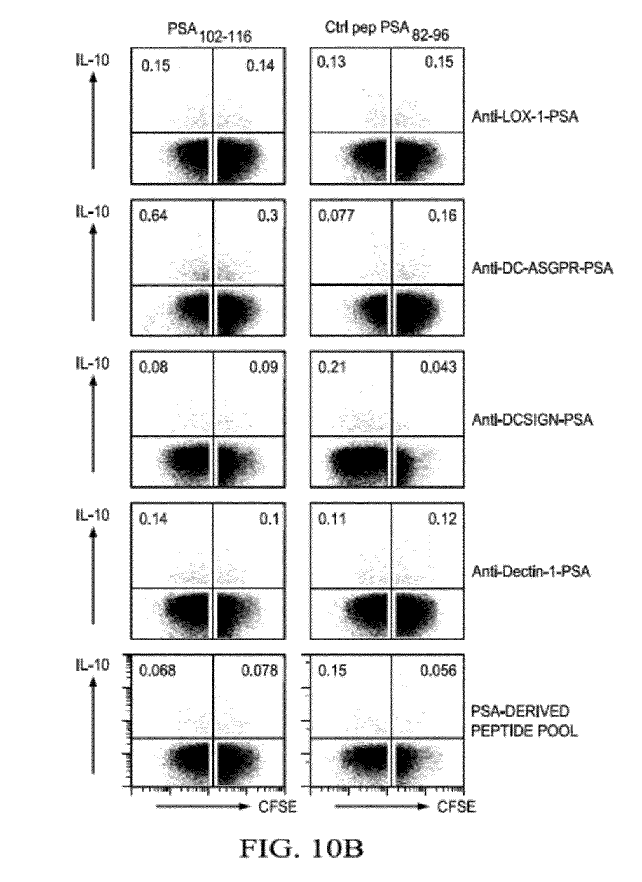

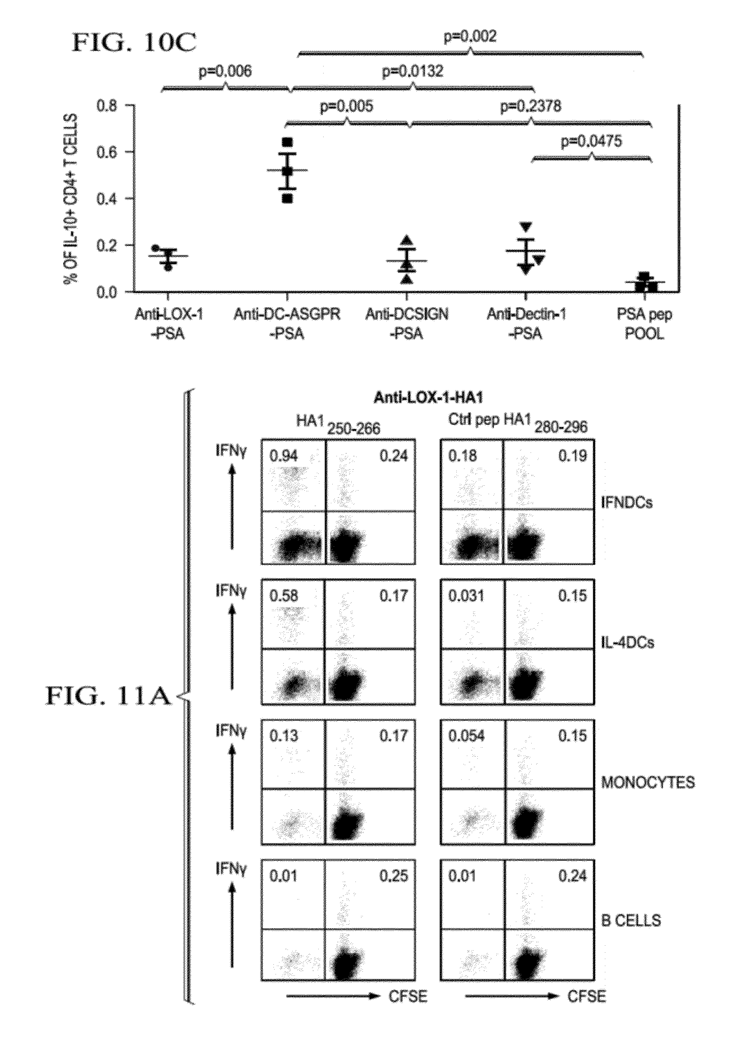

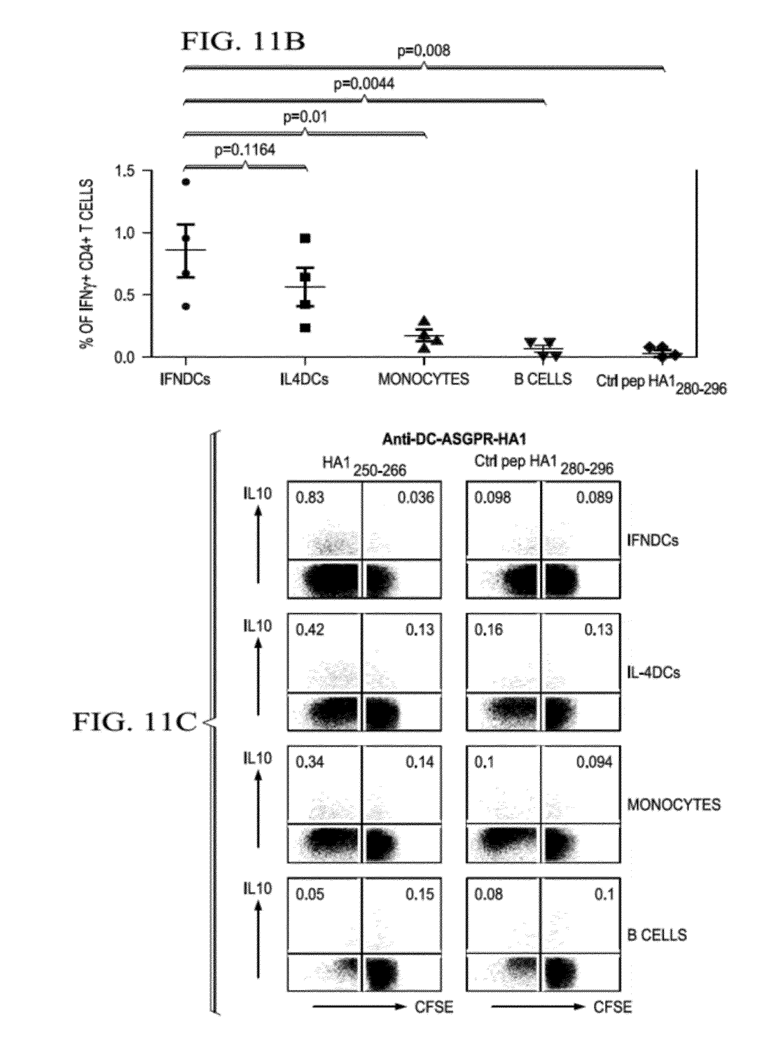

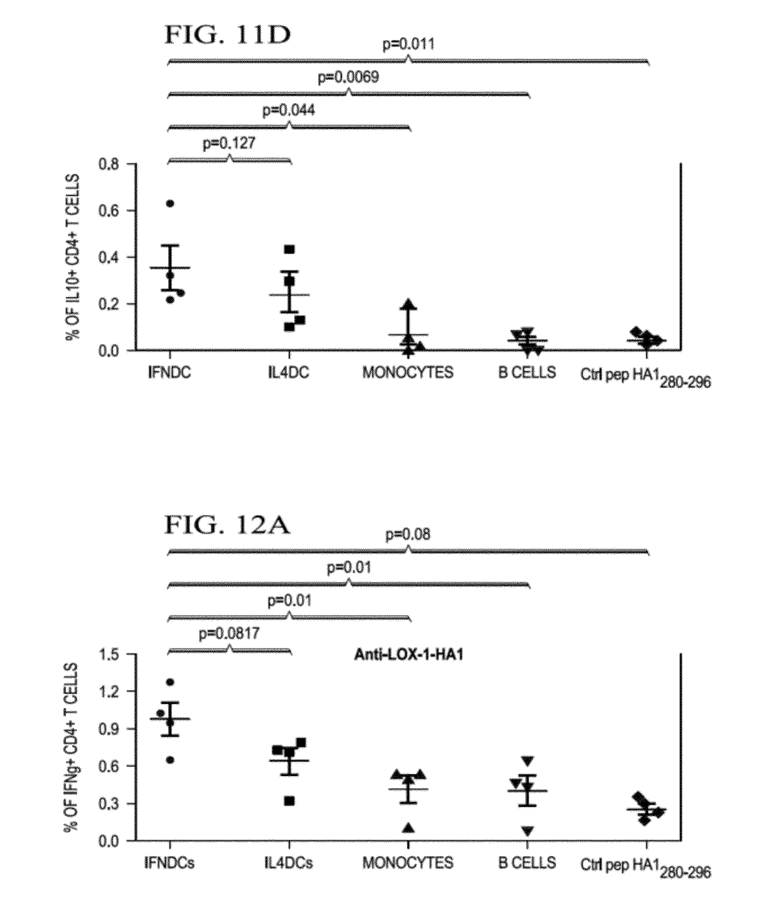

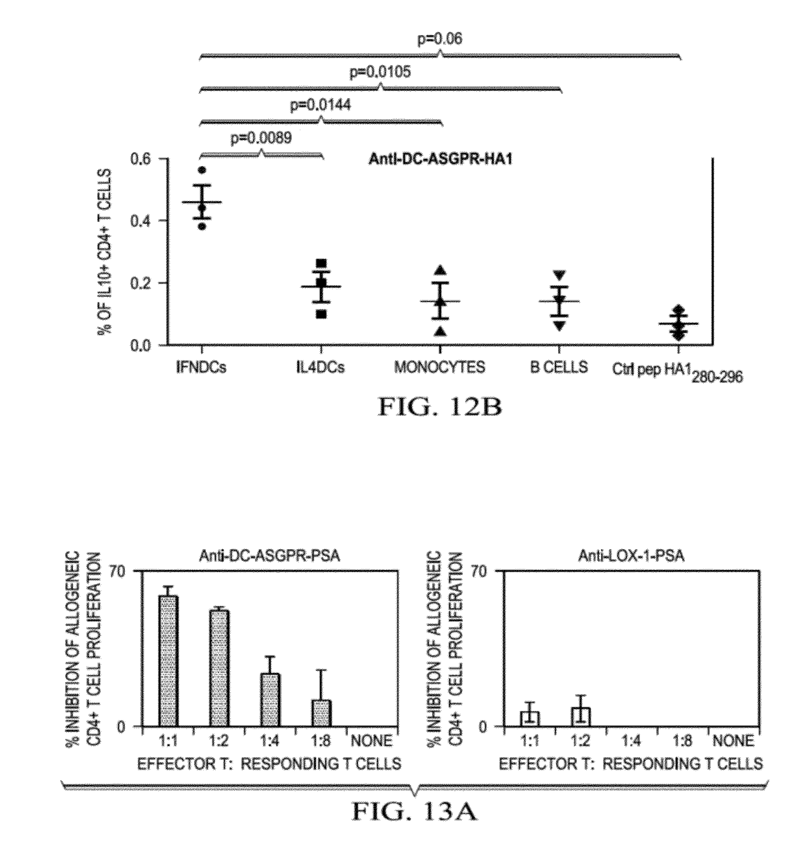

The inventors then tested whether self-antigens delivered to DCs via DC-ASGPR could result in antigen-specific IL-10 Tregs. Indeed, DCs loaded with anti-DC-ASGPR-PSA resulted in enhanced IL-10-producing PSA-specific CD4⁺ T-cell responses (FIGS. 5E, 5F and 8). In contrast, CD4⁺ T-cells induced with anti-LOX-1-PSA secreted higher levels of IFNγ than those induced with anti-DC-ASGPR-PSA (FIG. 5F). The majority of PSA-specific IL-10-producing CD4⁺ T-cells expressed IFNγ during restimulation with PMA/ionomycin (FIG. 5G). They also expressed CTLA-4, but not Foxp3 (FIG. 9). The present inventors further tested whether DC-SIGN^{12, 19} and Dectin-1²⁰ might also induce IL-10 Tregs (FIGS. 10A-10C). mAbs (anti-Dectin-1: 15E2 IgG2a and anti-DC-SIGN: 20B3 IgG1) were generated and fused to PSA as for anti-DC-ASGPR-PSA (not shown). Both anti-Dectin-1-PSA and anti-DC-SIGN-PSA induced PSA-specific IFNγ-producing CD4⁺ T-cells, but not significantly increased IL-10-producing CD4⁺ T-cells. Taken together, PSA targeting to DCs via DC-ASGPR can induce antigen-specific IL-10 Treg. PSA-derived peptides determined in this study are summarized in Table 2. Upon loading with anti-DC-ASGPR-HA1, DCs, particularly IFNDCs, were more efficient than monocytes and B cells in expanding both IFNγ- and IL-10-producing CD4⁺ T-cells (FIGS. 11A-11D). DCs loaded with peptides were also more potent than other APCs loaded with the same peptides to stimulate T-cells to express cytokines (FIGS. 12A and 12B).

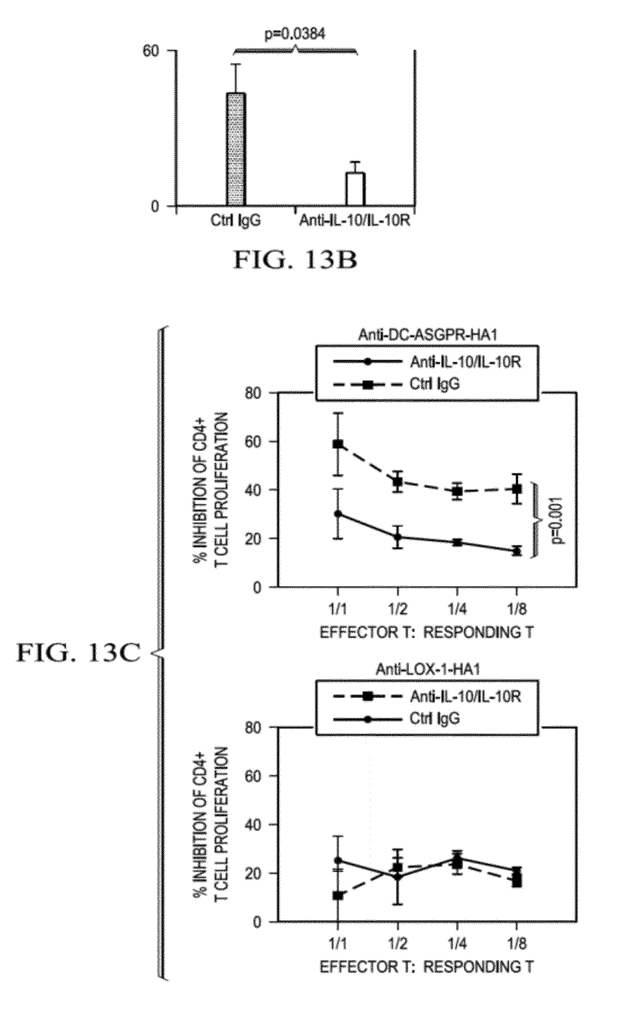

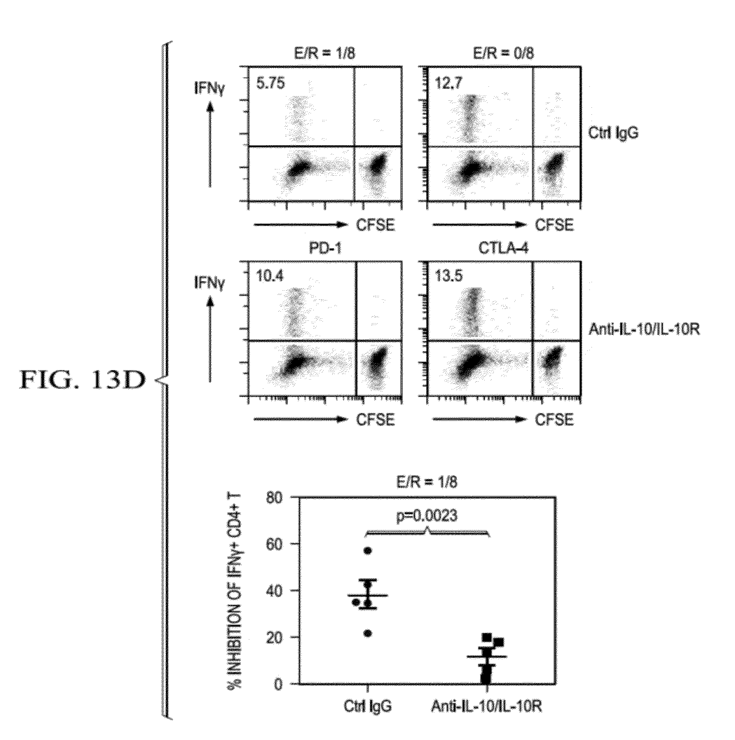

The suppressor function of CD4⁺ T-cells was assessed in both allogeneic and HA1-specific settings. From co-cultures of T-cells and IFNDCs loaded with anti-DC-ASGPR-PSA or anti-LOX-1-PSA, CFSE^{low}CD4⁺ T-cells were sorted. Increasing numbers of the sorted T-cells were added to the cultures of autologous IFNDCs and allogeneic CD4⁺ T-cells. CD4⁺ T-cells induced with anti-DC-ASGPR-PSA inhibited allogeneic CD4⁺ T-cell proliferation in a dose-dependent fashion (left panel in FIG. 13A). In contrast, CD4⁺ T-cells with anti-LOX-1-PSA did not significantly inhibit allogeneic T-cell proliferation (right panel in FIG. 13A). Neutralizing IL-10 reduced the inhibition of allogeneic CD4⁺ T-cell proliferation by T-cells induced with anti-DC-ASGPR-PSA (FIG. 13B). The CFSE^{low}CD4⁺ T-cells elicited by anti-DC-ASGPR-HA1, but not by anti-LOX-1-HA1, were also able to inhibit autologous T-cell proliferation in trans-wells (FIG. 13C). IFNγ expression induced by HA1-derived HA1₂₅₀₋₂₆₆-loaded DCs was also inhibited by the CFSE^{low}CD4⁺ T-cells expanded with anti-DC-ASGPR-HA1 (left panels in FIG. 13D),

TABLE 2
Predicted binding scores of PSA-derived peptides to corresponding HLA class II of the
healthy donors tested in this study.

| | | | Published peptides that |
|---|---|---|---|
| | | | are restricted to |
| | Class II | Peptides tested | MHC class II of donors |
| Donors | types | in this study/Binding Scores | tested in this study |
| Donor 1 | HLA-DRB01- | PSA₃₀₋₄₄ ECEKHSQPWQVLVAS (SEQ ID NO: 17)/ | HLA-DRB01-1501-restricted |
| | 1501 | (983.6) | peptides: |
| | | PSA₁₀₂₋₁₁₆ DMSLLKNRFLRPGDD (SEQ ID NO: 18)/ | (Clin Cancer Res 2005; 11(8) |
| | | (7.3) | 2853-2861) |
| | | PSA₁₆₂₋₁₇₆ PEEFLTPKKLQCVDL (SEQ ID NO: 19)/ | PSA₁₇₁₋₁₉₀ LQCVDLHVISNDVCAQVHPQ |
| | | (1000000.0) | (SEQ ID NO: 21) |
| | | PSA₈₂₋₉₆ HPEDIGQVFQVSHSF (SEQ ID NO: 20)/ | PSA₂₂₁₋₂₄₀ GVLQGITSWGSEPCALPERP |
| | | ((397.1) | (SEQ ID NO: 22) |
| | HLA-DRB1*14 | NA/NA | |
| | HLA-DQB1* | NA/NA | |
| Donor 2 | HLA-DRB01- | PSA₅₈₋₇₂ QWVLTAAHCIRNKSV (SEQ ID NO: 23)/ | |
| | 11 | (14859.9) | |
| | | PSA₇₈₋₉₂ HSLFHPEDTGQVFQV (SEQ ID NO: 24)/ | |
| | | (3293.1) | |
| | | PSA₁₄₆₋₁₆₀ PALGTTCYASGWGSI (SEQ ID NO: 25)/ | |
| | | (1000000.0) | |
| | | PSA₁₇₈₋₁₉₂ VISNDVCAQVHPQKV (SEQ ID NO: 26)/ | |
| | | (130615.6) | |
| | | PSA₂₂₆₋₂₄₀ ITSWGSEPCALPERP (SEQ ID NO: 27)/ | |
| | | (871249.4) | |
| | HLA-DRB5* | PSA₅₈₋₇₂ QWVLTAAHCIRNKSV (SEQ ID NO: 28)/ | |
| | | (146.1) | |
| | | PSA₇₈₋₉₂ HSLFHPEDTGQVFQV (SEQ ID NO: 29)/ | |
| | | (1000000.0) | |
| | | PSA₁₄₆₋₁₆₀ PALGTTCYASGWGSI (SEQ ID NO: 30)/ | |
| | | (1000000.0) | |
| | | PSA₁₇₈₋₁₉₂ VISNDVCAQVHPQKV (SEQ ID NO. 31)/ | |
| | | (6707.6) | |
| | | PSA₂₂₆₋₂₄₀ ITSWGSEPCALPERP (SEQ ID NO: 32)/ | |
| | | (1000000.0) | |
| | HLA-DRB3* | NA/NA | |
| | HLA-DRB16 | NA/NA | |
| Donor 3 | HLA-DRB01- | PSA₅₀₋₆₄ CGGVLVHPQWVLTAA (SEQ ID NO: 33)/ | HLA-DRB4 expressing |
| | 15XX | (356.0) | donors |
| | | PSA₆₂₋₇₆ TAAHCIRNKSVILLG (SEQ ID NO: 34)/ | (Clin Exp Immunnol 1998; |
| | | (1.4) | 114: 166-172) |
| | | PSA₆₆₋₈₀ CIRNKSVILLGRHSL (SEQ ID NO: 35)/ | PSA₄₉₋₆₃ ILLGRMSLFMPEDTG |
| | | (1.4) | (SEQ ID NO: 48) |
| | | PSA₁₄₆₋₁₆₀ PALGTTCYASGWGSI (SEQ ID NO: 36)/ | PSA₆₄₋₇₈ QVFQVSHSFPHPLYD |
| | | (1071.3) | (SEQ ID NO: 49) |
| | | PSA₁₆₆₋₁₈₀ LTPKKLQCVDLHVIS (SEQ 10 NO: 37)/ | PSA₉₅₋₁₀₉ NDLMLLRLSEPAELT |
| | | (438254.3) | (SEQ ID NO: 50) |
| | HLA-DPB01- | PSA₅₀₋₆₄ CGGVLVHPQWVLTAA (SEQ ID NO: 38)/ | PSA₁₂₂₋₁₃₆ PALGTTCVASGMGSI |
| | 07XX | (266306.1) | (SEQ ID NO: 51) |
| | | PSA₆₂₋₇₆ TAAHCIRNKSVILLG (SEQ ID NO: 39)/ | PSA₁₃₄₋₁₄₈ GSIEPEEFLTPDQMK |
| | | (14.9) | (SEQ ID NO: 52) |
| | | PSA₆₆₋₈₀ CIRNKSVILLGRHSL (SEQ ID NO: 40)/ | PSA₁₄₈₋₁₆₀ KKLQCVQLHVISM |
| | | (14.9) | (SEQ ID NO: 53) |
| | | PSA₁₄₆₋₁₆₀ PALGTTCYASGWGSI (SEQ ID NO: 41)/ | PSA₁₉₄₋₂₀₈ GPLVCNGVLQGITSM |
| | | (798.0) | (SEQ ID NO: 54) |
| | | PSA₁₆₆₋₁₈₀ LTPKKLQCVDLHVIS (SEQ ID NO: 42)/ | PSA₂₀₀₋₂₁₄ GVLQGITSMGSEPCA |
| | | (183.7) | (SEQ ID NO: 55) |
| | HLA-DRB5* | PSA₅₀₋₆₄ CGGVLVHPQWVLTAA (SEQ ID NO. 43)/ | |
| | | (6279.2) | |
| | | PSA₆₂₋₇₆ TAAHCIRNKSVILLG (SEQ ID NO: 44)/ | |
| | | (434.6) | |
| | | PSA₆₆₋₈₀ CIRNKSVILLGRHSL (SEQ ID NO: 45)/ | |
| | | (1.5) | |
| | | PSA₁₄₆₋₁₆₀ PALGTTCYASGWGSI (SEQ ID NO: 46)/ | |
| | | (1000000.0) | |
| | | PSA₁₆₆₋₁₈₀ LTPKKLQCVDLHVIS (SEQ ID NO: 47)/ | |
| | | (1408.0) | |
| | HLA-DRB4* | NA/NA | |
| | HLA-DQB1* | NA/NA | |

1. Peptides were predicted by the algorithm in the Web: (http://tools.immuneepitope.org/analyze/cgi-bin/mhc_II_binding.py). Maximum and minimal ABR score is 1000000 and 0, respectively.
2. NA: alleles are not available in the algorithm

This inhibition was recovered by blocking IL-10 (FIG. 13D). Thus, IL-10 secreted from the CD4⁺ T-cells suppresses T-cell proliferation and IFNγ expression.

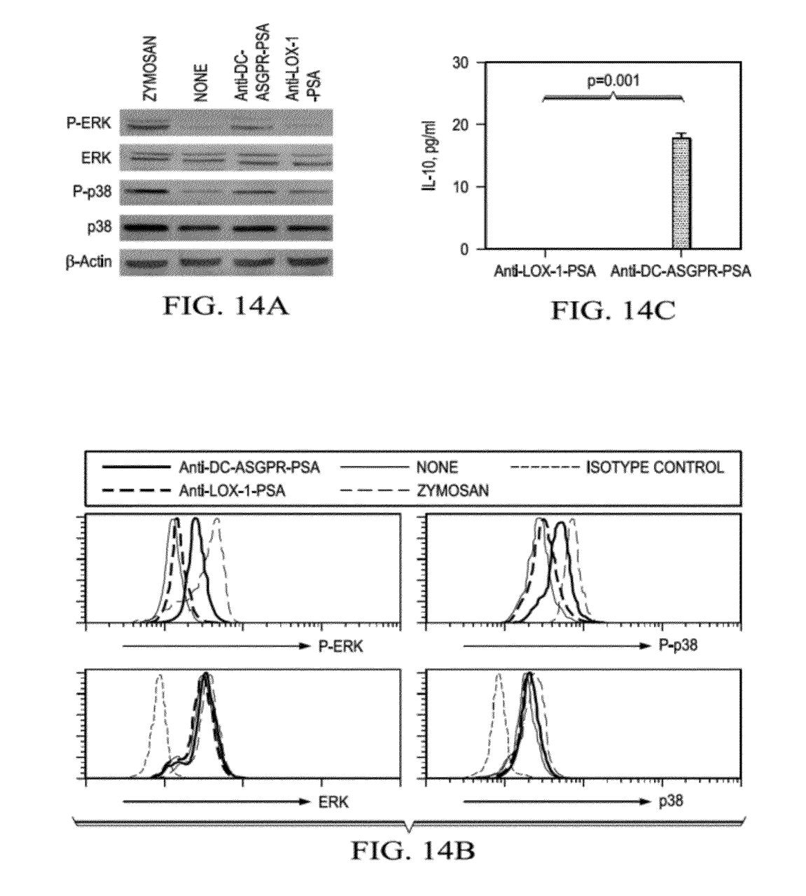

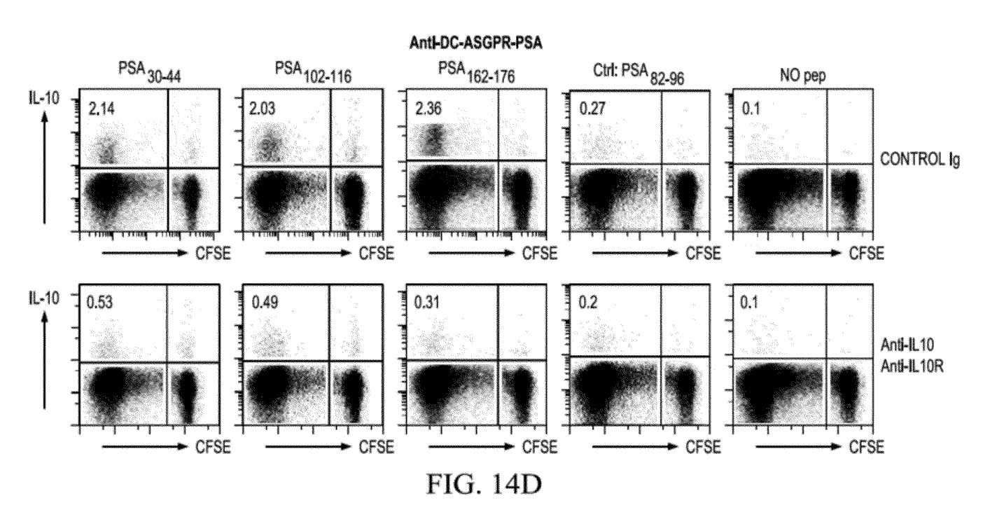

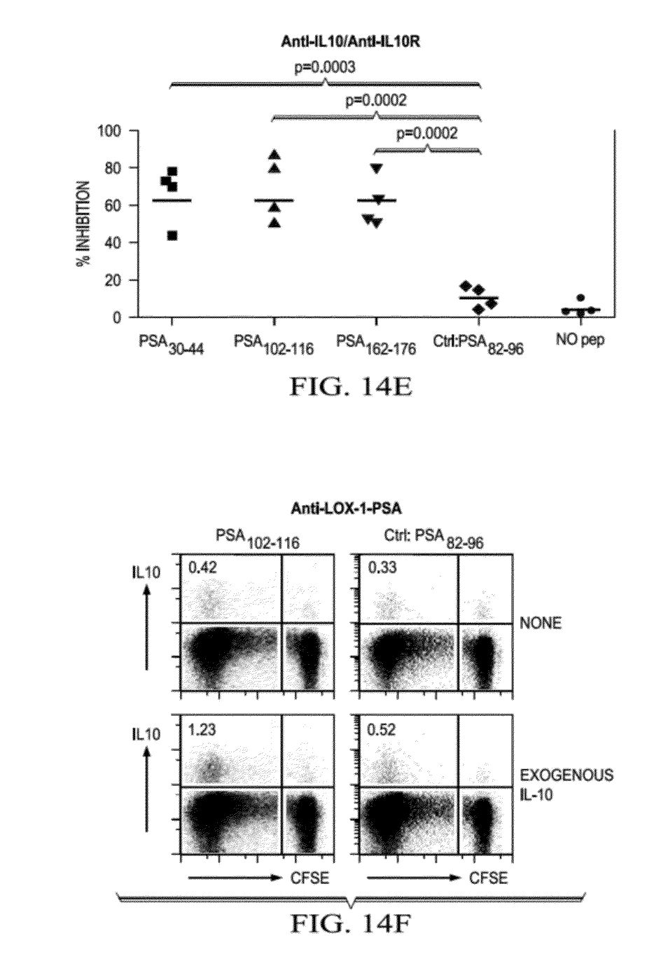

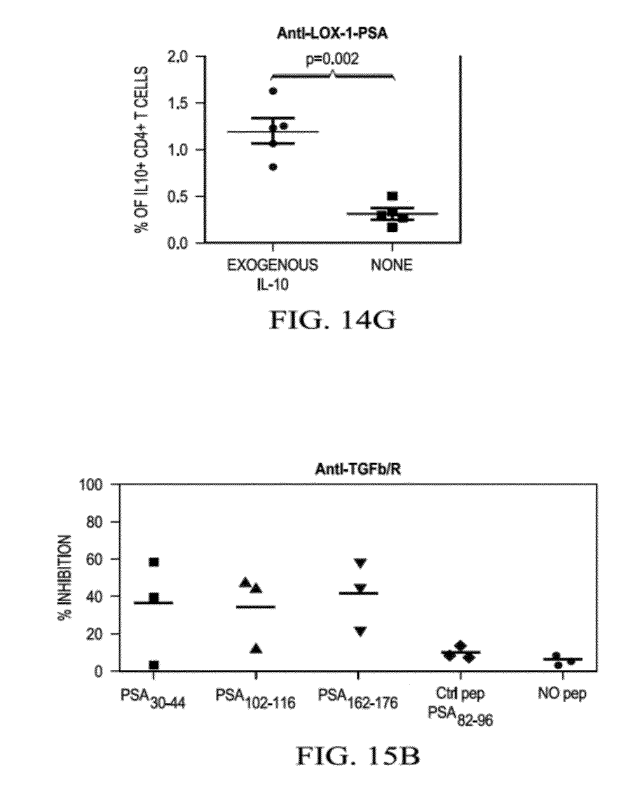

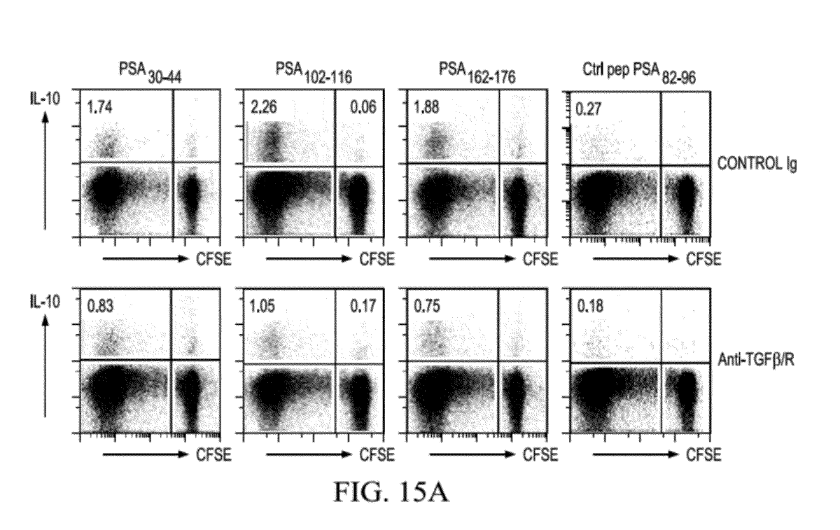

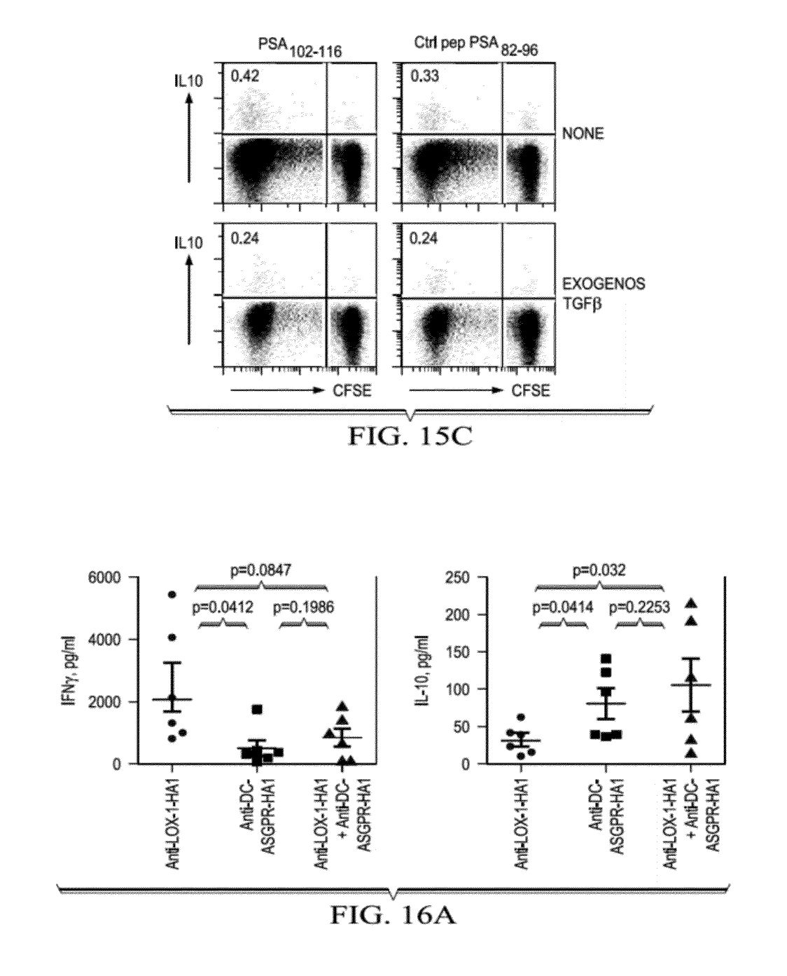

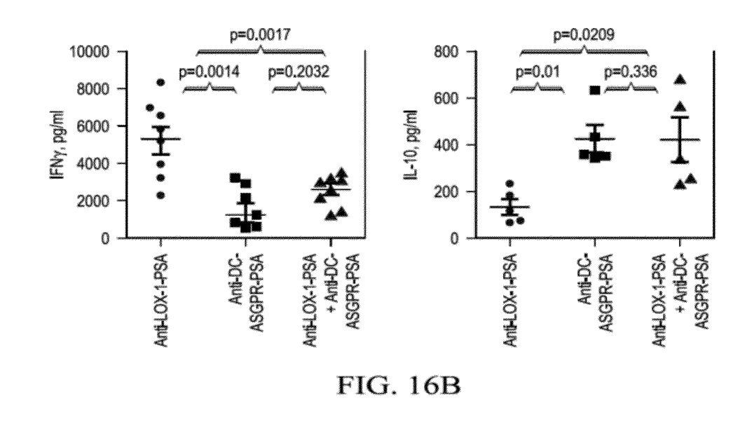

The activation of mitogen-activated protein kinase (MAPK) is directly associated with IL-10 induction in DCs^{21, 22}. Furthermore, differences in IL-10 production by DCs correlates with the strength of extracellular signal-related kinases (ERK) activation²². DCs exposed to anti-ASGPR-PSA displayed enhanced phosphorylation of ERK as well as p38 (FIG. 14A). Phosphorylation of ERIC/p38 was further demonstrated by flow cytometry (FIG. 14B). Consistently, DCs exposed to anti-DC-ASGPR-PSA, but not anti-LOX-1-PSA, secrete IL-10 (FIG. 14C). Anti-DC-ASGPR-PSA did not induce IL-27 or ICOSL expression (not shown) that can contribute to IL-10 secretion from T-cells^{23, 24}. Blocking IL-10 in co-cultures of CD4⁺ T-cells and DCs loaded with anti-DC-ASGPR-PSA resulted in a decreased induction of PSA-specific IL-10-producing CD4⁺ T-cells (FIGS. 14D and 14E). Conversely, the addition of exogenous IL-10 into the co-cultures of CD4⁺ T-cells and DCs loaded with anti-LOX-1-PSA enhanced IL-10-producing CD4⁺ T-cell responses (FIGS. 14F and 14G). Neutralizing TGFβ also resulted in slightly reduced IL-10-producing CD4⁺ T-cell responses, but exogenous TGFβ did not enhance IL-10-producing CD4⁺ T-cell responses (FIGS. 15A-15C). Taken together, our data demonstrate that delivering PSA to DCs via DC-ASGPR, but not LOX-1, induce ERK/p38-phosphorylation and IL-10 expression in DCs, which contributes to the induction of IL-10 Tregs. Interestingly, FIGS. 16A and 16B suggests that the biological function of DC-ASGPR could prevail over the LOX-1. Antigens delivered to DCs via DC-ASGPR and LOX-1 simultaneously resulted in decreased IFNγ-producing and enhanced IL-10-producing CD4⁺ T-cell responses.

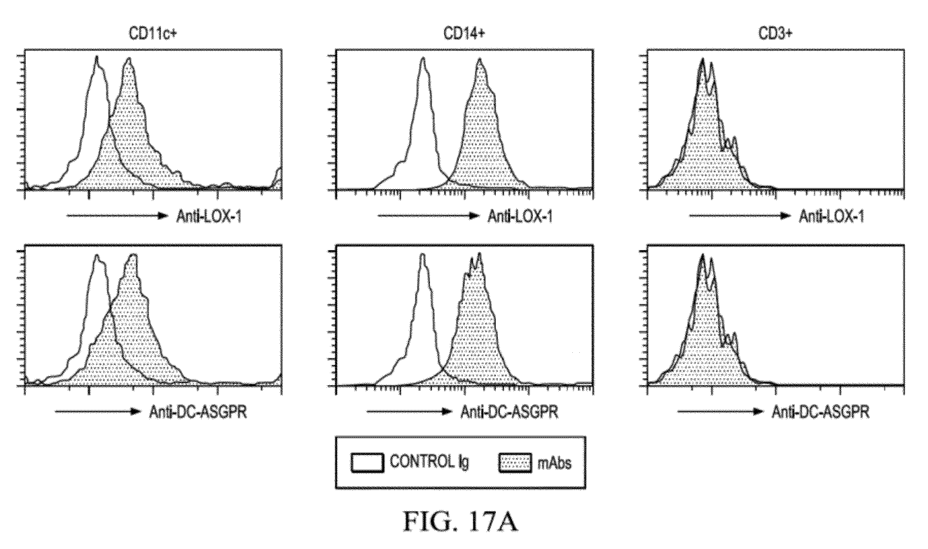

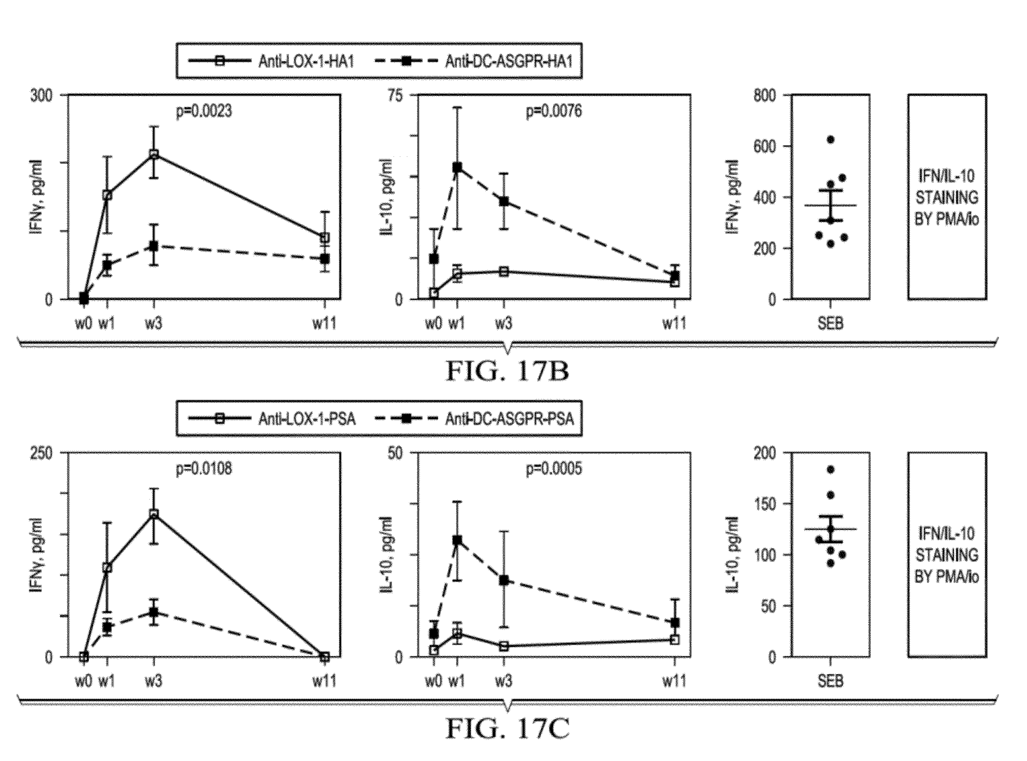

To extend the in vitro observations of the present invention in vivo, cynomolgous macaques were immunized with the fusion proteins. Both anti-LOX-1 and anti-DC-ASGPR mAbs bound to CD11c⁺ and CD14⁺ cells, but not CD3⁺ T-cells (FIG. 17A). All animals (a total 12 animals: 6 animals per group) were pre-immunized with live influenza viruses (H1N1, PR8). Sera displayed high levels of HA1-specific IgG (not shown). Four months after priming, animals were immunized with either anti-LOX-1-HA1 (right arm) and anti-LOX-1-PSA (left arm) or anti-DC-ASGPR-HA1 (right arm) and anti-DC-ASGPR-PSA (left arm). After three immunizations with recombinant fusion proteins, blood was collected as indicated in FIGS. 17B and 17C. PBMCs from animals immunized with anti-LOX-1-HA1 secreted significantly higher levels of IFNγ in response to HA1 peptide pools than animals immunized with anti-DC-ASGPR-HA1 (FIG. 17B). In contrast, PBMCs from animals immunized with anti-DC-ASGPR-HA1 secreted higher levels of IL-10 in response to HA1 peptides when compared to those immunized with anti-LOX-1-HA1. The same observation was made with animals that were immunized with PSA fusion proteins. IL-10-producing PSA-specific cellular responses were preferentially mounted in animals immunized with anti-DC-ASGPR-PSA, whereas animals immunized with anti-LOX-1-PSA preferentially mounted higher IFNγ-producing PSA-specific cellular responses (FIG. 17C). For both HA1 and PSA, the peak of IL-10-producing cellular responses was obtained at week one, but the peak of IFNγ-producing cellular responses was obtained at week three after the second boosting.

Given the crucial roles of IL-10 Tregs of Th1 origin in limiting host immune pathologies^{1-3, 5, 7, 8, 25-29}, these findings provide novel therapeutics for curing human diseases caused by non-recessive inflammatory responses. The in vivo establishment of antigen-specific IL-10 Tregs is an alternative to the current therapy approaches, such as repeated peptide immunizations^{6, 30} and adoptive transfers of in vitro generated IL-10 Tregs²⁹. The unique capacity of DC-ASGPR applies to both self and foreign antigens as well as naïve and memory CD4⁺ T-cell responses. Thus, DC-ASGPR also appears to be a universal target for designing vaccines against autoimmune diseases where autoantigens are defined, such as type 1 diabetes and multiple sclerosis.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application Publication No. 2008/0206262: Agents that Engage Antigen-Presenting Cells through Dendritic Cell Asialoglycoprotein Receptor (DC-ASGPR).
U.S. Patent Application Publication No. 2008/0241170: Vaccines based on Targeting Antigen to DCIR Expressed on Antigen-Presenting Cells.
1. Trinchieri, G. Interleukin-10 Production by Effector T Cells: Th1 Cells Show Self Control. J Exp Med 204, 239-43 (2007).
2. Groux, H Et Al. A CD4+ T-Cell Subset Inhibits Antigen-Specific T-Cell Responses and Prevents Colitis. Nature 389, 737-42 (1997).
3. Moore, K. W., De Waal Malefyt, R., Coffman, R. L. & O'Garra, A. Interleukin-10 and the Interleukin-10 Receptor. Annu Rev Immunol 19, 683-765 (2001).
4. Haringer, B., Lozza, L., Steckel, B. & Geginat, J. Identification and Characterization of IL-10/IFN-Gamma-Producing Effector-Like T Cells with Regulatory Function in Human Blood. J Exp Med 206, 1009-17 (2009).
5. Anderson, C. F., Oukka, M., Kuchroo, V. J. & Sacks, D. CD4(+)CD25(−)Foxp3(−) Th1 Cells are the Source of IL-10-Mediated Immune Suppression in Chronic Cutaneous Leishmaniasis. J Exp Med 204, 285-97 (2007).
6. Gabrysova, L. Et Al. Negative Feedback Control of the Autoimmune Response through Antigen-Induced Differentiation of IL-10-Secreting Th1 Cells. J Exp Med 206, 1755-67 (2009).
7. Jankovic, D. Et Al. Conventional T-Bet(+)Foxp3(−) Th1 Cells are the Major Source of Host-Protective Regulatory IL-10 During Intracellular Protozoan Infection. J Exp Med 204, 273-83 (2007).
8. O'Garra, A. & Vieira, P. T(H)1 Cells Control Themselves by Producing Interleukin-10. Nat Rev Immunol 7, 425-8 (2007).
9. Banchereau, J. & Steinman, R. M. Dendritic Cells and the Control of Immunity. Nature 392, 245-52 (1998).
10. Brown, G. D. Dectin-1: A Signalling Non-TLR Pattern-Recognition Receptor. Nat Rev Immunol 6, 33-43 (2006).
11. Figdor, C. G., Van Kooyk, Y. & Adema, G. J. C-Type Lectin Receptors on Dendritic Cells and Langerhans Cells. Nat Rev Immunol 2, 77-84 (2002).
12. Geijtenbeek, T. B. & Gringhuis, S. I. Signaling through C-Type Lectin Receptors: Shaping Immune Responses. Nat Rev Immunol 9, 465-79 (2009).
13. Akira, S., Takeda, K. & Kaisho, T. Toll-Like Receptors: Critical Proteins Linking Innate and Acquired Immunity. Nat Immunol 2, 675-80 (2001).
14. Valladeau, J. Et Al. Immature Human Dendritic Cells Express Asialoglycoprotein Receptor Isoforms for Efficient Receptor-Mediated Endocytosis. J Immunol 167, 5767-74 (2001).
15. Delneste, Y. Et Al. Involvement Of LOX-1 In Dendritic Cell-Mediated Antigen Cross-Presentation. Immunity 17, 353-62 (2002.)
16. Geijtenbeek, T. B. Et Al. Mycobacteria Target DC-SIGN to Suppress Dendritic Cell Function. J Exp Med 197, 7-17 (2003).
17. Reddy, M. P. Et Al. Elimination of Fc Receptor-Dependent Effector Functions of A Modified IGg4 Monoclonal Antibody to Human CD4. J Immunol 164, 1925-33 (2000).
18. Macian, F. Et Al. Transcriptional Mechanisms Underlying Lymphocyte Tolerance. Cell 109, 719-31 (2002).
19. Smits, H. H. Et Al. Selective Probiotic Bacteria Induce IL-10-Producing Regulatory T Cells In Vitro by Modulating Dendritic Cell Function through Dendritic Cell-Specific Intercellular Adhesion Molecule 3-Grabbing Nonintegrin. J Allergy Clin Immunol 115, 1260-7 (2005).
20. Dillon, S. Et Al. Yeast Zymosan, A Stimulus for TLR2 and Dectin-1, Induces Regulatory Antigen-Presenting Cells and Immunological Tolerance. J Clin Invest 116, 916-28 (2006).
21. Agrawal, A., Dillon, S., Denning, T. L. & Pulendran, B. ERK1−/− Mice Exhibit Th1 Cell Polarization and Increased Susceptibility to Experimental Autoimmune Encephalomyelitis. J Immunol 176, 5788-96 (2006).
22. Kaiser, F. Et Al. TPL-2 Negatively Regulates Interferon-Beta Production in Macrophages and Myeloid Dendritic Cells. J Exp Med 206, 1863-71 (2009).
23. Ito, T. Et Al. Plasmacytoid Dendritic Cells Prime IL-10-Producing T Regulatory Cells by Inducible Costimulator Ligand. J Exp Med 204, 105-15 (2007).
24. Iwasaki, A., Torres, C. A. T., Ohashi, P. S., Robinson, H. L. & Barber, B. H. The Dominant Role of Bone Marrow-Derived Cells in CTL Induction Following Plasmid DNA Immunization at Different Sites1. J. Immunol. 159, 1-4 (1997).
25. Gazzinelli, R. T. Et Al. In the Absence of Endogenous IL-10, Mice Acutely Infected with Toxoplasma gondii Succumb to A Lethal immune Response Dependent on CD4+ Cells and Accompanied by Overproduction of IL-12, IFN-Gamma and TNF-Alpha. J Immunol 157, 798-805 (1996).
26. Allan, S. E. Et Al. CD4+ T-Regulatory Cells: Toward Therapy for Human Diseases. Immunol Rev 223, 391-421 (2008).
27. Burkhart, C., Liu, G. Y., Anderton, S. M., Metzler, B. & Wraith, D. C. Peptide-Induced T Cell Regulation of Experimental Autoimmune Encephalomyelitis: A Role For IL-10. Int Immunol 11, 1625-34 (1999).
28. O'Neill, E. J., Day, M. J. & Wraith, D. C. is Essential for Disease Protection Following Intranasal Peptide Administration in the C57BL/6 Model of EAE. J Neuroimmunol 178, 1-8 (2006).
29. Roncarolo, M. G. & Battaglia, M. Regulatory T-Cell immunotherapy for Tolerance to Self Antigens and Alloantigens in Humans. Nat Rev Immunol 7, 585-98 (2007).
30. Sundstedt, A., O'Neill, E. J., Nicolson, K. S. & Wraith, D. C. Role for IL-10 in Suppression Mediated by Peptide-Induced Regulatory T Cells In Vivo. J Immunol 170, 1240-8 (2003).

What is claimed is:

1. A method for generating one or more of antigen-specific regulatory T-cells (Tregs) comprising:
isolating one or more human dendritic cells (DCs) from a subject;
loading one or more antigens into the one or more DCs with an anti-DC-asialoglycoprotein receptor (DC-ASGPR) specific antibody or binding fragment thereof conjugated or fused to the one or more antigens to form antigen-loaded DCs; and
contacting the antigen-loaded DCs with one or more naïve T-cells, wherein the antigen-loaded DCs stimulate the proliferation of one or more antigen-specific Tregs.

2. The method of claim 1, wherein the one or more antigens comprise peptides or proteins.

3. The method of claim 2, wherein the peptide is a foreign or a self-antigen.

4. The method of claim 2, wherein the peptide triggers an allergic or asthmatic response.

5. The method of claim 1, wherein the one or more antigens comprises a bacterial; a viral; a fungal; a protozoan; or a cancer protein.

6. The method of claim 1, wherein the antigens comprise HA-1, PSA, or combinations and modifications thereof.

7. The method of claim 1, wherein the one or more antigen-specific Tregs are IL-10 secreting Tregs.

8. The method of claim 1, wherein the dendritic cells are used for a prophylaxis; a treatment; amelioration of symptoms; or any combinations thereof of one or more self-antigen mediated autoimmune diseases; multiple sclerosis; influenza; or cancer.

9. The method of claim 1, wherein the autoimmune diseases are selected from the group consisting of allergies; asthma; Coeliac disease; diabetes mellitus type 1 (IDDM); systemic lupus erythematosus (SLE); Sjögren's syndrome; Churg-Strauss Syndrome; Hashimoto's thyroiditis; Graves disease; idiopathic thrombocytopenic purpura; graft rejection; multiple sclerosis; psoriasis; and rheumatoid arthritis (RA).

10. The method of claim 1, wherein the antibody is made by a hybridoma cell contained in ATCC Deposit No. PTA-10248.

11. A vaccine composition against one or more autoantigen mediated autoimmune diseases comprising:
an anti-DC-asialoglycoprotein receptor (ASGPR) specific antibody or binding fragment thereof conjugated to, or fused to, one or more autoantigens; and
one or more optional pharmaceutically acceptable adjuvants, wherein the vaccine composition generates, enhances the production, or both of one or more autoantigen specific, IL-10 secreting regulatory T-cells (Tregs).

12. The composition of claim 11, wherein the autoimmune diseases are selected from the group consisting of allergies; asthma; Coeliac disease; diabetes mellitus type 1 (IDDM); systemic lupus erythematosus (SLE); Sjögren's syndrome; Churg-Strauss Syndrome; Hashimoto's thyroiditis; Graves disease; idiopathic thrombocytopenic purpura; graft rejection; multiple sclerosis; psoriasis; and rheumatoid arthritis (RA).

13. The composition of claim 11, wherein the Tregs are autoantigen-specific IL-10 Tregs.

14. The composition of claim 11, wherein the vaccine is administered orally, parenterally or intra-nasally.

15. The composition of claim 11, wherein the one or more antigens comprise peptides; proteins; lipid, carbohydrate; nucleic acid; and combinations thereof.

16. The composition of claim 11, wherein the composition binds to and activates dendritic cells that activate the IL-10 secreting Tregs.

17. The composition of claim 11, wherein the antibody is made by a hybridoma cell contained in ATCC Deposit No. PTA-10248.

18. A method for treating, for prophylaxis or for amelioration of symptoms of a cancer in a subject comprising the steps of:
identifying the subject in need for the treatment, prophylaxis, or the amelioration of symptoms against a cancer;
administering a therapeutically effective amount of a pharmaceutical composition or a vaccine in an amount sufficient to treat, for the prophylaxis or amelioration of the symptoms, wherein the composition comprises: a recombinant fusion protein of an anti-DC-asialoglycoprotein receptor (ASGPR) specific antibody or binding fragment thereof conjugated or fused to one or more cancer specific antigens and one or more optional pharmaceutically acceptable excipients or adjuvants.

19. The method of claim 18, wherein the composition or the vaccine is administered orally, parenterally, or intra-nasally.

20. The method of claim 18, wherein the composition generates, enhances a level, or both of one or more of regulatory T-cells (Tregs) that are specific to a prostate serum antigen (PSA).

21. The method of claim 20, wherein the antigen is a prostate cancer antigen and the Tregs are prostate cancer-specific IL-10 Tregs.

22. The method of claim 20, wherein the cancer specific antigens are peptides selected from tumor associated antigens are selected from CEA; prostate; prostate specific antigen (PSA); HER-2/neu; BAGE; GAGE; MAGE 1-4; 6 and 12; MUC (Mucin) (e.g. MUC-1, MUC-2, etc.); GM2 and GD2 gangliosides; ras; myc; tyrosinase; MART (melanoma antigen); MARCO-MART; cyclin B1; cyclin D; Pmel 17 (gp 100); GnT-V intron V sequence (N-acetylglucoaminyltransferase V intron V sequence); Prostate Ca psm; prostate serum antigen (PSA); PRAME (melanoma antigen); β-catenin; (melanoma ubiquitous mutated gene product); GAGE (melanoma antigen) 1; BAGE (melanoma antigen) 2-10; c-ERB2 (Her2/neu); EBNA (Epstein-Barr Virus nuclear antigen) 1-6; gp75; human papilloma virus (HPV) E6 and E7; p53; lung resistance protein (LRP); Bcl-2; and Ki-67.

23. The method of claim 18, wherein the antibody is made by a hybridoma cell contained in ATCC Deposit No. PTA-10248.

24. A method for treating, for prophylaxis or for amelioration of symptoms of a pathogen in a subject comprising the steps of:
identifying the subject in need for the treatment, prophylaxis, or the amelioration of symptoms against the pathogen;
administering a therapeutically effective amount of a pharmaceutical composition or a vaccine in an amount sufficient to treat, for the prophylaxis, or amelioration of the symptoms, wherein the composition comprises: a recombinant fusion protein of an anti-DC-asialoglycoprotein receptor (ASGPR) specific antibody or binding fragment thereof conjugated or fused to one or more pathogenic antigens and one or more optional pharmaceutically acceptable excipients or adjuvants.

25. The method of claim 24, wherein the vaccine is administered orally, parenterally, or intra-nasally.

26. The method of claim 24, wherein the vaccine generates, enhances a level, or both of one or more of pathogen-specific regulatory T-cells (Tregs).

27. The method of claim 24, wherein the antibody is made by a hybridoma cell contained in ATCC Deposit No. PTA-10248.

28. A method for treating, for prophylaxis, or amelioration of symptoms of autoantigen mediated autoimmune diseases in a subject comprising the steps of:
identifying the subject in need of the treatment, the prophylaxis or the amelioration of the symptoms of the autoimmune disease; and
administering a therapeutically effective amount of vaccine comprising a recombinant fusion protein of an anti-DC-asialoglycoprotein receptor (ASGPR) specific antibody or binding fragment thereof conjugated or fused to one or more autoantigens, and one or more optional pharmaceutically acceptable adjuvants, wherein the vaccine composition generates, enhances the production, or both of one or more autoantigen specific regulatory T-cells (Tregs).

29. The method of claim 28, wherein the autoimmune diseases are selected from the group consisting of allergies; asthma; Coeliac disease; diabetes mellitus type 1 (IDDM); systemic lupus erythematosus (SLE); Sjögren's syndrome; Churg-Strauss Syndrome; Hashimoto's thyroiditis; Graves' disease; idiopathic thrombocytopenic purpura; graft rejection; multiple sclerosis; psoriasis; and rheumatoid arthritis (RA).

30. The method of claim 28, wherein the autoimmune disease is diabetes mellitus type 1 (IDDM).

31. The method of claim 28, wherein the Tregs are autoantigen-specific Tregs that secrete IL-10.

32. The method of claim 28, wherein the composition or the vaccine is administered orally, parenterally, or intra-nasally.

33. The method of claim 28, wherein the one or more antigens comprise peptides, proteins, lipid, carbohydrate, nucleic acid, and combinations thereof.

34. The method of claim 28, wherein the antibody is made by a hybridoma cell contained in ATCC Deposit No. PTA-10248.

35. A pharmaceutical composition for generating self-antigen specific regulatory T-cells (Tregs) comprising:
a recombinant fusion protein of an anti-DC-asialoglycoprotein receptor (ASGPR) specific antibody or binding fragment thereof conjugated or fused to one or more self-antigens; and
one or more optional pharmaceutically acceptable excipients or adjuvants.

36. The composition of claim 35, wherein the composition is used for a prophylaxis, a treatment, amelioration of symptoms of one or more autoantigen mediated autoimmune diseases; multiple sclerosis; influenza; or cancer.

37. The composition of claim 35, wherein the self-antigen is selected from antigens that cause allergies; asthma; Coeliac disease; diabetes mellitus type 1 (IDDM); systemic lupus erythematosus (SLE); Sjögren's syndrome; Churg-Strauss Syndrome; Hashimoto's thyroiditis; Graves' disease; idiopathic thrombocytopenic purpura; graft rejection; multiple sclerosis; psoriasis; and rheumatoid arthritis (RA).

38. The composition of claim 35, wherein the antibody is made by a hybridoma cell contained in ATCC Deposit No. PTA-10248.
